(19) United States
(12) Patent Application Publication
(10) Pub. No.: US 2017/0071569 A1
(43) Pub. Date: Mar. 16, 2017

(54) ULTRASONIC DIAGNOSTIC APPARATUS AND SIGNAL PROCESSING APPARATUS

(71) Applicant: Toshiba Medical Systems Corporation, Otawara-shi (JP)

(72) Inventor: Takeshi SATO, Nasushiobara (JP)

(73) Assignee: Toshiba Medical Systems Corporation, Otawara-shi (JP)

(21) Appl. No.: 15/257,459

(22) Filed: Sep. 6, 2016

(30) Foreign Application Priority Data
Sep. 14, 2015 (JP) 2015-181125

(51) Int. Cl. A61B 8/14; G01S 7/52; A61B 8/06; A61B 8/00; A61B 8/08

(52) U.S. Cl. CPC A61B 8/14; A61B 8/4444; A61B 8/465; A61B 8/467; A61B 8/5207; A61B 8/488; A61B 8/06; A61B 8/54; A61B 8/08; A61B 8/5276; G01S 7/5205; G01S 7/52033

(57) ABSTRACT

An ultrasonic diagnostic apparatus according to an embodiment includes determination circuitry and generation circuitry. The determination circuitry determines whether or not a reflected wave signal received by each channel of an ultrasonic probe is saturated. The generation circuitry generates reflected wave data through a phasing addition process using an output signal for which a predetermined process corresponding to a result of the determination acquired by the determination circuitry is performed.

### CROSS-REFERENCE TO RELATED APPLICATIONS

This application is based upon and claims the benefit of priority from Japanese Patent Application No. 2015-181125, filed on Sep. 14, 2015; the entire contents of which are incorporated herein by reference.

### FIELD

Embodiments described herein relate generally to an ultrasonic diagnostic apparatus and a signal processing apparatus.

### BACKGROUND

In recent years, by performing plane wave transmission or transmission covering a wide range similar to the plane wave transmission, all received rasters within an ultrasonic wave frame can be acquired in real time at one-time transmission. Here, this will be referred to as all-raster parallel simultaneous reception. By applying the all-raster parallel simultaneous reception to a blood flow imaging method using data between frames, a blood flow display system capable of detecting a low speed to a high speed in high frame rate display can be built. Since a transmission interval for a blood flow and a frame period match each other, high frame rate display and a high instant speed are secured, and an observation time of infinity can be acquired. Accordingly, a steep moving target indicator (MTI) filter having a low cutoff frequency can be configured, and detection can be performed up to a low-speed blood flow while suppressing low-speed clutter.

### BRIEF DESCRIPTION OF THE DRAWINGS

FIG. 1 is a block diagram that illustrates an example of the configuration of an ultrasonic diagnostic apparatus according to a first embodiment;

FIG. 2 is a diagram that illustrates an example of a case where blood flow information is power-displayed;

FIG. 3 is a diagram that illustrates an example of a sound field in general ultrasonic wave transmission;

FIG. 4 is a diagram that illustrates an example of a sound field in plane wave transmission;

FIG. 5A is a diagram that illustrates an example of RF signals in a distance direction acquired in a case where a point reflector moves in a direction separating from an ultrasonic beam, and reflected wave signals of all channels CH are not saturated;

FIG. 5B is a diagram that illustrates an example of IQ signals in a distance direction acquired in a case where a point reflector moves in a direction separating from an ultrasonic beam, and reflected wave signals of all channels CH are not saturated;

FIG. 5C is a diagram that illustrates an example of IQ signals in a Doppler direction acquired in a case where a point reflector moves in a direction separating from an ultrasonic beam, and reflected wave signals of all channels CH are not saturated;

FIG. 5D is a diagram that illustrates an example of a Doppler shift acquired in a case where a point reflector moves in a direction separating from an ultrasonic beam, and reflected wave signals of all channels CH are not saturated;

FIG. 5E is a diagram that illustrates an example of RF signals in a distance direction acquired in a case where a point reflector moves in a direction separating from an ultrasonic beam, and reflected wave signals of some channels CH are saturated;

FIG. 5F is a diagram that illustrates an example of IQ signals in a distance direction acquired in a case where a point reflector moves in a direction separating from an ultrasonic beam, and reflected wave signals of some channels CH are saturated;

FIG. 5G is a diagram that illustrates an example of IQ signals in a Doppler direction acquired in a case where a point reflector moves in a direction separating from an ultrasonic beam, and reflected wave signals of some channels CH are saturated;

FIG. 5H is a diagram that illustrates an example of a Doppler shift acquired in a case where a point reflector moves in a direction separating from an ultrasonic beam, and reflected wave signals of some channels CH are saturated;

FIG. 6A is a diagram that illustrates an example of RF signals in a distance direction acquired in a case where a point reflector moves to traverse an ultrasonic beam at a high speed, and reflected wave signals of all channels CH are not saturated;

FIG. 6B is a diagram that illustrates an example of IQ signals in a distance direction acquired in a case where a point reflector moves to traverse an ultrasonic beam at a high speed, and reflected wave signals of all channels CH are not saturated;

FIG. 6C is a diagram that illustrates an example of IQ signals in a Doppler direction acquired in a case where a point reflector moves to traverse an ultrasonic beam at a high speed, and reflected wave signals of all channels CH are not saturated;

FIG. 6D is a diagram that illustrates an example of a Doppler shift acquired in a case where a point reflector moves to traverse an ultrasonic beam at a high speed, and reflected wave signals of all channels CH are not saturated;

FIG. 6E is a diagram that illustrates an example of RF signals in a distance direction acquired in a case where a point reflector moves to traverse an ultrasonic beam at a high speed, and reflected wave signals of some channels CH are saturated;

FIG. 6F is a diagram that illustrates an example of IQ signals in a distance direction acquired in a case where a point reflector moves to traverse an ultrasonic beam at a high speed, and reflected wave signals of some channels CH are saturated;

FIG. 6G is a diagram that illustrates an example of IQ signals in a Doppler direction acquired in a case where a point reflector moves to traverse an ultrasonic beam at a high speed, and reflected wave signals of some channels CH are saturated;

FIG. 6H is a diagram that illustrates an example of a Doppler shift acquired in a case where a point reflector moves to traverse an ultrasonic beam at a high speed, and reflected wave signals of some channels CH are saturated;

FIG. 7 is a block diagram that illustrates an example of the configuration of reception circuitry according to the first embodiment;

FIG. 8 is a diagram that illustrates an effect of the ultrasonic diagnostic apparatus according to the first embodiment;

FIG. 9 is a block diagram that illustrates an example of the configuration of reception circuitry according a second embodiment;

FIG. 10 is a diagram that illustrates an example of reception circuitry and a beam former according to a third embodiment;

FIG. 11 is a diagram that illustrates the third embodiment;

FIG. 12 is a diagram that illustrates a fourth embodiment;

FIG. 13 is a block diagram that illustrates an example of the configuration of reception circuitry and Doppler processing circuitry according to a fifth embodiment;

FIG. 14 is a block diagram that illustrates an example of the configuration of reception circuitry and B-mode processing circuitry according to a sixth embodiment; and

FIG. 15 is a block diagram that illustrates an example of the configuration of reception circuitry according to a modified example of the sixth embodiment.

### DETAILED DESCRIPTION

Hereinafter, ultrasonic diagnostic apparatuses and signal processing apparatuses according to embodiments will be described with reference to the drawings. However, the embodiments are not limited to the following embodiments. In principle, a content described in one embodiment is similarly applied to the other embodiments.

An ultrasonic diagnostic apparatus according to an embodiment includes determination circuitry and generation circuitry. The determination circuitry determines whether or not a reflected wave signal received by each channel of an ultrasonic probe is saturated. The generation circuitry generates reflected wave data through a phasing addition process using an output signal for which a predetermined process corresponding to a result of the determination acquired by the determination circuitry is performed.

### First Embodiment

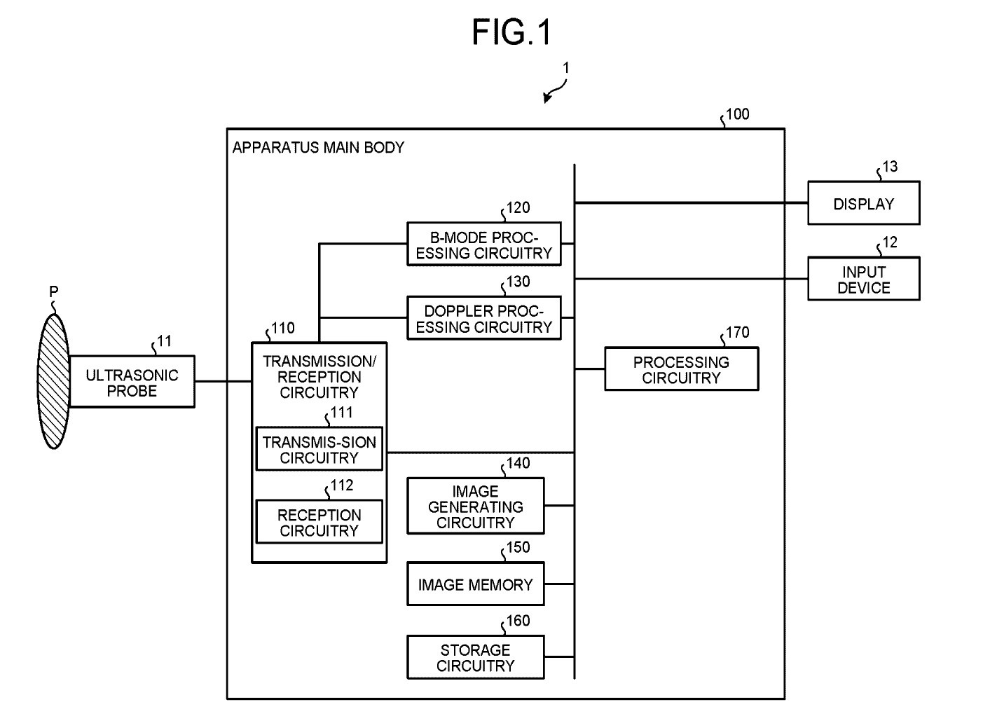

FIG. 1 is a block diagram that illustrates an example of the configuration of an ultrasonic diagnostic apparatus 1 according to a first embodiment. As illustrated in FIG. 1, the ultrasonic diagnostic apparatus 1 according to the first embodiment includes: an ultrasonic probe 11; an input device 12; a display 13; and an apparatus main body 100. The ultrasonic probe 11 is communicably connected to transmission/reception circuitry 110 included in the apparatus main body 100 to be described later. In addition, the input device 12 and the display 13 are communicably connected to various circuitry included in the apparatus main body 100.

The ultrasonic probe 11 is brought into contact with a body surface of a subject P and transmits and receives ultrasonic waves. For example, the ultrasonic probe 11 includes a plurality of piezoelectric transducer elements (also referred to as transducer elements). The plurality of piezoelectric transducer elements generate ultrasonic waves based on a transmission signal supplied from the transmission/reception circuitry 110. The generated ultrasonic waves are reflected on an in-body tissue of the subject P and are received by the plurality of piezoelectric transducer elements as reflected wave signals. The ultrasonic probe 11 transmits the reflected wave signals received by the plurality of piezoelectric transducer elements to the transmission/reception circuitry 110.

In the first embodiment, the ultrasonic probe 11 may be a 1D array probe that scans (two-dimension scanning) a two-dimensional area inside a subject P or a mechanical 4D probe or a 2D array probe that scans (three-dimensional scanning) a three-dimensional area inside a subject P so as to be applicable.

The input device 12, for example, corresponds to a mouse, a keyboard, a button, a panel switch, a touch command screen, a foot switch, a trackball, a joystick, or the like. The input device 12 receives various setting requests from an operator of the ultrasonic diagnostic apparatus 1 and appropriately transmits the received various setting requests to circuitry of the apparatus main body 100.

The display 13 displays a graphical user interface (GUI) used for operator's inputting various setting requests using the input device 12 or displays an image (ultrasonic wave image) based on ultrasonic wave image data generated by the apparatus main body 100 or the like.

The apparatus main body 100 is a device that generates ultrasonic wave image data based on reflected wave signals received by the ultrasonic probe 11. As illustrated in FIG. 1, the apparatus main body 100, for example, includes: transmission/reception circuitry 110; B-mode processing circuitry 120; a Doppler processing circuitry 130; an image generating circuitry 140; an image memory 150; storage circuitry 160; and processing circuitry 170. The transmission/reception circuitry 110, the B-mode processing circuitry 120, the Doppler processing circuitry 130, the image generating circuitry 140, the image memory 150, the storage circuitry 160; and the processing circuitry 170 are connected to be communicable with each other.

The transmission/reception circuitry 110 controls the transmission/reception of ultrasonic waves using the ultrasonic probe 11. For example, the transmission/reception circuitry 110 includes transmission circuitry 111 and reception circuitry 112 and controls the transmission/reception of ultrasonic waves performed by the ultrasonic probe 11 based on an instruction from the processing circuitry 170 to be described later. The transmission circuitry 111 generates transmission waveform data and generates a transmission signal used for the ultrasonic probe 11 to transmit an ultrasonic wave based on the generated transmission waveform data. Then, the transmission circuitry 111 applies the transmission signal to the ultrasonic probe 11, thereby transmitting an ultrasonic beam in which the ultrasonic wave converges in a beam shape.

For example, the transmission circuitry 111 causes the ultrasonic probe 11 to perform ultrasonic wave scanning transmitting a plane wave under the control of the processing circuitry 170. In addition, the transmission circuitry 111 causes the ultrasonic probe 11 to perform ultrasonic wave scanning receiving reflected wave signals in a plurality of scanning lines.

In addition, the transmission circuitry 111 causes the ultrasonic probe 11 to perform ultrasonic wave scanning using a data row between frames as a Doppler data row under the control of the processing circuitry 170 (see Japanese Patent No. 3724846 and Japanese Patent Application Publication No. 2014-42823). For example, the transmission circuitry 111 causes the ultrasonic probe 11 to perform first ultrasonic wave scanning acquiring information relating to a motion of a moving body within a first scanning range and causes the ultrasonic probe 11 to perform ultrasonic wave scanning of each of a plurality of divided ranges acquired by dividing a second scanning range as second ultrasonic wave scanning acquiring information of the shape of a tissue within the second scanning range in a time divisional manner during the first ultrasonic wave scanning under the control of the processing circuitry 170.

Furthermore, the transmission circuitry 111 causes the ultrasonic probe 11 to perform ultrasonic wave scanning having a first transmission ultrasonic wave and a second transmission ultrasonic wave acquired by inverting the phase of the first transmission ultrasonic wave as one set under the control of the processing circuitry 170.

The reception circuitry 112 generates reflected wave data in which a reflection component reflected from a direction corresponding to the reception directivity of a reflected wave signal is emphasized by performing an addition process with a predetermined delay time applied to the reflected wave signal received by the ultrasonic probe 11 and transmits the generated reflected wave data to the B-mode processing circuitry 120 and the Doppler processing circuitry 130.

For example, the reception circuitry 112 includes: amplification circuitry (described as an “Amp” as is appropriate); an analog/digital (A/D) converter (described as an “ADC” as is appropriate); generation circuitry; quadrature detection circuitry (described as an “IQ” as is appropriate); and the like. The amplification circuitry performs a gain correction process by amplifying a reflected wave signal for each channel. The A/D converter performs an A/D conversion of the gain-corrected reflected wave signals.

The generation circuitry applies a reception delay time that is necessary for determining the reception directivity to digital data. Then, the generation circuitry performs an addition process of adding the reflected wave signals for which the reception delay time has been applied. According to the addition process performed by the generation circuitry, reflection components, which are reflected from a direction corresponding to the reception directivity, of the reflected wave signals are emphasized.

Then, the quadrature detection circuitry converts an output signal of the adder into an in-phase signal (I signal, I: in-phase) and a quadrature signal (Q signal, Q: quadrature phase) of a baseband. Then, the quadrature detection circuitry stores the I signal and the Q signal (hereinafter, referred to as IQ signals) in a buffer as reflected wave data. In addition, the quadrature detection circuitry may convert the output signal of the adder into a radio frequency (RF) signal and then store the RF signal stored in a buffer. The IQ signals and the RF signal are signals (reception signals) in which phase information is included. While the quadrature detection circuitry has been described to be arranged on a later stage of the generation circuitry, the embodiment is not limited thereto. For example, the quadrature detection circuitry may be arranged on a former stage of the generation circuitry. In such a case, the generation circuitry performs an addition process of adding an I signal and a Q signal.

The B-mode processing circuitry 120 performs various kinds of signal processing for the reflected wave data generated based on the reflected wave signals by the reception circuitry 112. The B-mode processing circuitry 120 performs logarithmic amplification, an envelope detection process, and the like for the reflected wave data received from the reception circuitry 112 and generates data (B mode data) in which a signal intensity for each sample point (observation point) is represented as the brightness of luminance. The B-mode processing circuitry 120 transmits the generated B mode data to the image generating circuitry 140.

In addition, the B-mode processing circuitry 120 performs signal processing used for performing harmonic imaging that images harmonic components. As the harmonic imaging, contrast harmonic imaging (CHI) and tissue harmonic imaging (THI) are known. In addition, in the contrast harmonic imaging or the tissue harmonic imaging, as a scanning system, amplitude modulation (AM), phase modulation (PM) called a “pulse subtraction method” or a “pulse inversion method”, and AMPM capable of acquiring both the effects of the AM and the effects of the FM by combining the AM and the PM are known.

The Doppler processing circuitry 130 generates data (Doppler data) acquired by extracting motion information based on the Doppler effect of a moving body from the reflected wave data received from the reception circuitry 112 as sample points within the scanning area. More specifically, the Doppler processing circuitry 130 generates Doppler data acquired by extracting an average speed, a variance value, a power value, and the like as motion information of the moving body as sample points. Here, the moving body, for example, is a blood flow, a tissue such as a cardiac wall, or an imaging agent. The Doppler processing circuitry 130 transmits the generated Doppler data to the image generating circuitry 140.

The image generating circuitry 140 generates ultrasonic wave image data based on the data generated by the B-mode processing circuitry 120 and the Doppler processing circuitry 130. For example, the image generating circuitry 140 generates B-mode image data representing the intensity of a reflected wave as luminance based on the B mode data generated by the B-mode processing circuitry 120. In addition, the image generating circuitry 140 generates Doppler image data representing moving body information based on the Doppler data generated by the Doppler processing circuitry 130. This Doppler image data is speed image data, variance image data, power image data, or image data combining these.

The image memory 150 is a memory that stores data generated by the B-mode processing circuitry 120, the Doppler processing circuitry 130, and the image generating circuitry 140. For example, the image memory 150 stores the ultrasonic wave image data generated by the image generating circuitry 140 in association with an electrocardiographic waveform of a subject P. In a case where the amount of data stored in the image memory 150 exceeds the storage capacity of the image memory 150, data is erased in order of old data to new data, and the image memory is updated.

The storage circuitry 160 is a storage device that stores various kinds of data. For example, the storage circuitry 160 stores control programs used for the transmission/reception of ultrasonic waves, image processing, and a display process, diagnosis information (for example, a patient ID, a doctor's opinion, or the like), and various kinds of data such as a diagnosis protocol and various kinds of body marks. In addition, the data stored in the storage circuitry 160 may be transmitted to an external device through an interface unit not illustrated in the drawing.

In addition, the storage circuitry 160 stores data stored by the B-mode processing circuitry 120, the Doppler processing circuitry 130, and the image generating circuitry 140. For example, the storage circuitry 160 stores ultrasonic wave image data corresponding to a predetermined heart rate designated by an operator. In addition, the storage circuitry 160 is an example of a storage unit that stores a plurality of images acquired by scanning a subject P for a predetermined period.

The processing circuitry 170 controls the whole process of the ultrasonic diagnostic apparatus 1. More specifically, the processing circuitry 170 controls the processes of the transmission/reception circuitry 110, the B-mode processing circuitry 120, the Doppler processing circuitry 130, the image generating circuitry 140, and the like based on various kinds of setting requests input from an operator through the input device 12 and various kinds of control programs and various kinds of data read from the storage circuitry 160. In addition, the processing circuitry 170 displays the ultrasonic wave image data stored in the image memory 150 on the display 13.

For example, the processing circuitry 170 causes the ultrasonic probe 11 to perform ultrasonic wave scanning transmitting a plane wave by controlling the transmission circuitry 111. In addition, for example, the processing circuitry 170 causes the ultrasonic probe 11 to perform ultrasonic wave scanning receiving reflected wave signals in a plurality of scanning lines by controlling the transmission circuitry 111. Furthermore, for example, the processing circuitry 170, by controlling the transmission circuitry 111, causes the ultrasonic probe 11 to perform first ultrasonic wave scanning acquiring information relating to a motion of a moving body within a first scanning range and causes the ultrasonic probe 11 to perform ultrasonic wave scanning of each of a plurality of divided ranges acquired by dividing a second scanning range during the first ultrasonic wave scanning in a time divisional manner as second ultrasonic wave scanning acquiring information of the shape of a tissue within the second scanning range. In addition, for example, the processing circuitry 170, by controlling the transmission circuitry ill, causes the ultrasonic probe 11 to perform ultrasonic wave scanning having a first transmission ultrasonic wave and a second transmission ultrasonic wave acquired by inverting the phase of the first transmission ultrasonic wave as one set.

A plurality of constituent elements illustrated in FIG. 1 may be integrated into one processor so as to realize the functions thereof. The term “processor” used in the description presented above, for example, represents a central processing unit (CPU), a graphics processing unit (GPU), or a circuit such as an application specific integrated circuit (ASIC), a programmable logic device (for example, a simple programmable logic device (SPLD), a complex programmable logic device (CPLD), and a field programmable gate array (FPGA)), or the like. The function of the processor is realized by reading and executing a program stored in the storage circuitry 160. Instead of storing the program in the storage circuitry 160, the program may be directly built in circuitry of the processor. In such a case, as the processor reads and executes the program built in the circuitry of the processor, the function thereof is realized. Each processor according to this embodiment is not limited to being configured as a single circuit for each processor, but the function thereof may be realized by configuring one processor by combining a plurality of independent circuits.

In the ultrasonic diagnostic apparatus 1 configured in this way, there are cases where all reception rasters within an ultrasonic wave frame are acquired in real time through one-time transmission by performing plane wave transmission or transmission covering a wide range similar thereto. Such ultrasonic wave scanning will be referred to as “all-raster parallel simultaneous reception”.

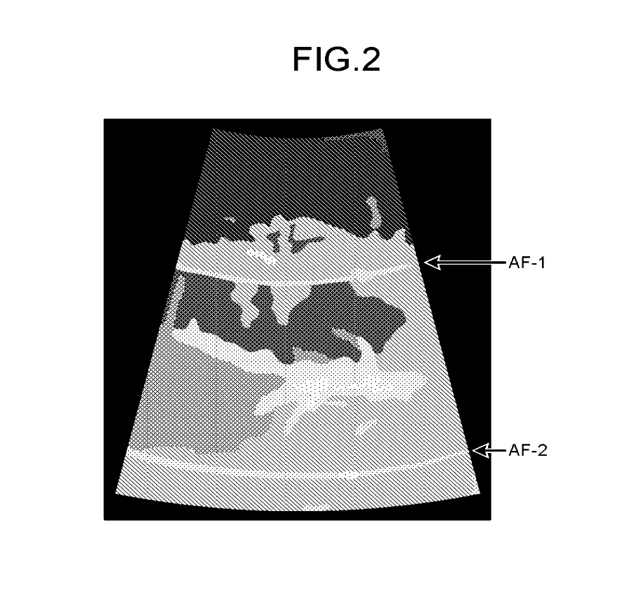

However, in a case where power display of a blood flow is performed by actually using “plane wave transmission+all-raster parallel simultaneous reception” in a living body, there are cases where an artifact is generated in a circular arc shape including a strong reflector. A problem that such artifacts are generated will be described with reference to FIG. 2. FIG. 2 is a diagram that illustrates an example of a case where blood flow information is power-displayed. As illustrated in FIG. 2, there are cases where artifacts having circular arc shapes as denoted by AF-1 and AF-2 are generated. The artifacts AF-1 and AF-2 are generated in a case where reflected wave signals from some elements disposed within paths of reflected wave signals from many ultrasonic transducer elements are greatly saturated, and signals from a strong reflector abruptly change.

In a case where the level of a minute blood flow signal according to a scattering echo from a red blood cell is set to 0 dB, a specular reflection echo from a vessel wall or a diaphragm is 100 dB or more. The value of a reflected wave signal of a strong reflector such as the vessel wall or the diaphragm exceeds far beyond the limit (generally, about 60 dB) of the dynamic range of a reflected wave signal from one element of the ultrasonic diagnostic apparatus 1. For this reason, a reflected wave signal from the vessel wall or the diaphragm is saturated mainly by amplification circuitry of the reception circuitry 112. In a color Doppler mode, in order to acquire a minute blood flow signal having a good S/N ratio, in a normal reception condition, the gain of the amplification circuitry is set to be high. For this reason, a reflected wave signal from a strong reflector is saturated. Since the saturation of the reflected wave signal occurs before beam forming, by observing reflected wave data after the beam forming, it cannot be recognized whether or not the reflected wave signal is saturated.

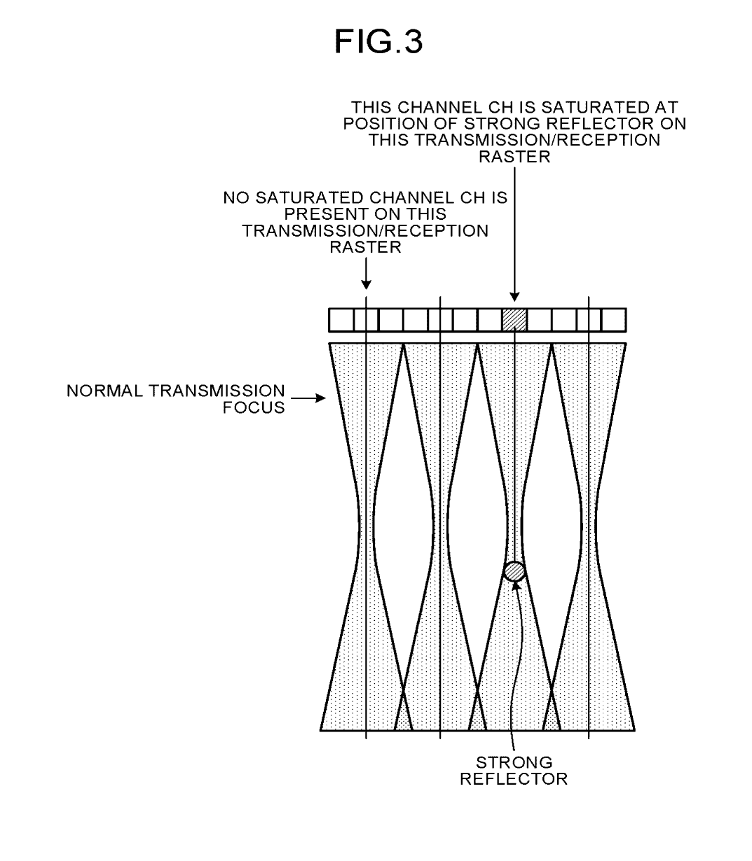

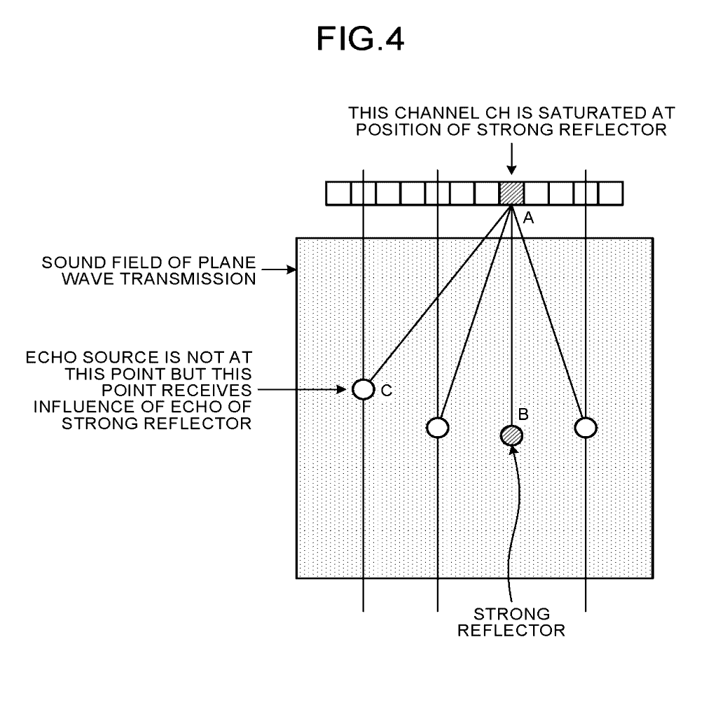

In case of the plane wave transmission+the all-raster parallel simultaneous reception, a reason for the generation of an artifact having a circular arc shape as illustrated in FIG. 2 will be described. FIG. 3 is a diagram that illustrates an example of a sound field in general ultrasonic wave transmission, and FIG. 4 is a diagram that illustrates an example of a sound field in plane wave transmission.

In the general ultrasonic wave transmission illustrated in FIG. 3, a transmission focus is applied on a same raster as a reception raster of an ultrasonic wave, and one raster is received for one-time transmission. In other words, since a focus is applied for both transmission and reception, a side lobe level of the transmission/reception sound field is low, and only reflected waves of a reflector that is present approximately on the raster are received. Even in a case where a reflected wave signal of a specific channel (CH) is saturated by a reflected wave signal from a strong reflector, the influence of the saturation is limited only to the place thereof. In such a case, even when a blood flow signal is erroneously recognized and displayed, it is well known that a strong reflector is incorrectly displayed as a blood flow signal, and thus, there is no serious problem. In addition, the speed may be calculated, and, based on a logic not displaying a signal in case of a low speed, the strong reflector may be configured not be displayed.

However, in case of the plane wave transmission illustrated in FIG. 4, a focus is not applied to the transmission, and a beam is narrowed only through a reception focus. For this reason, when a side lobe level of transmission/reception becomes high, a strong reflector is present at a point B as illustrated in FIG. 4, and a signal level of the depth of the point B is saturated in an element A that is disposed right above the point B, a signal level at a position that is located at a same distance as the point B from the element A, for example, a point C increases as well. In case of blood flow imaging, an MTI filter is present, and thus, only in a case where only the side lobe level is high, imaging is not performed. However, in a case illustrated as below, imaging is performed.

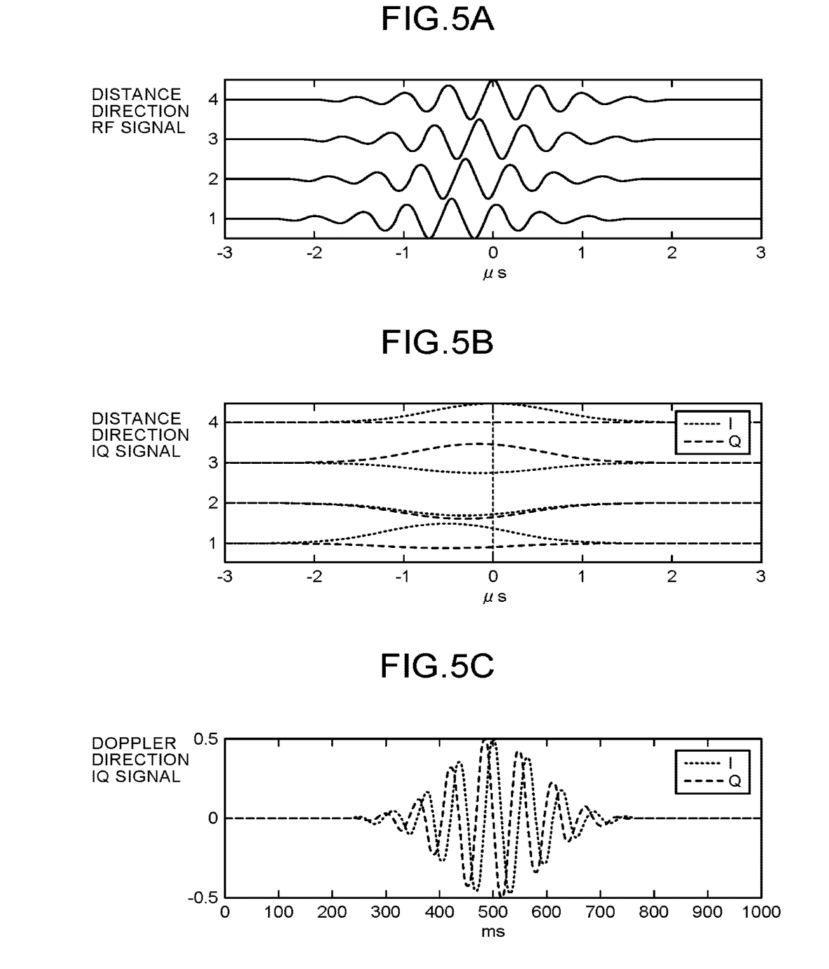

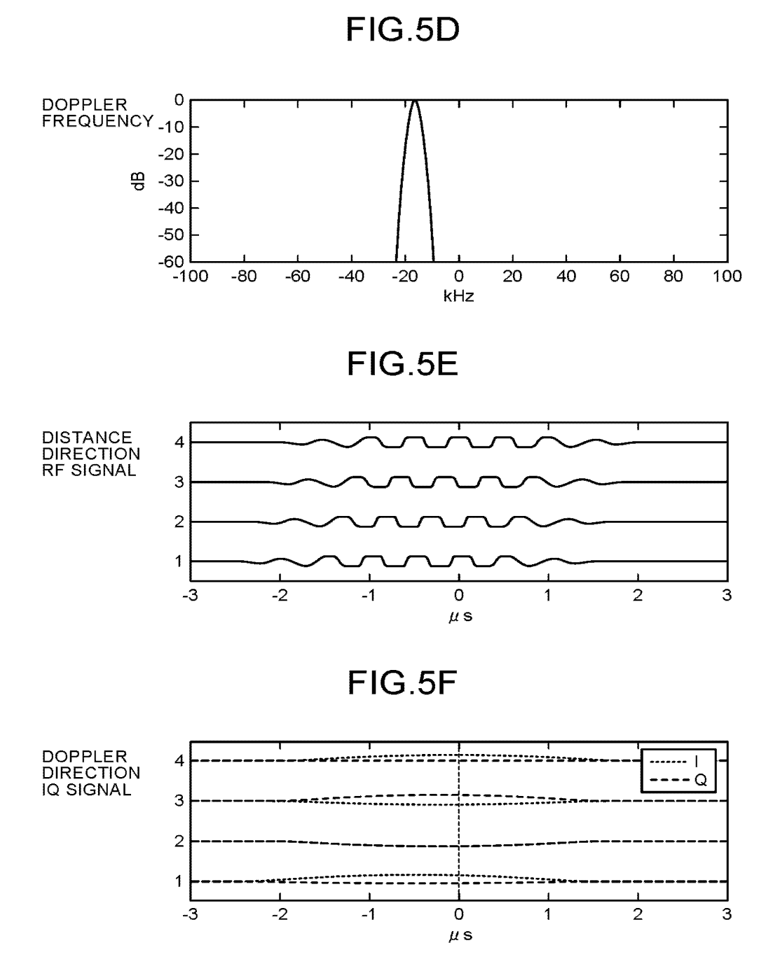

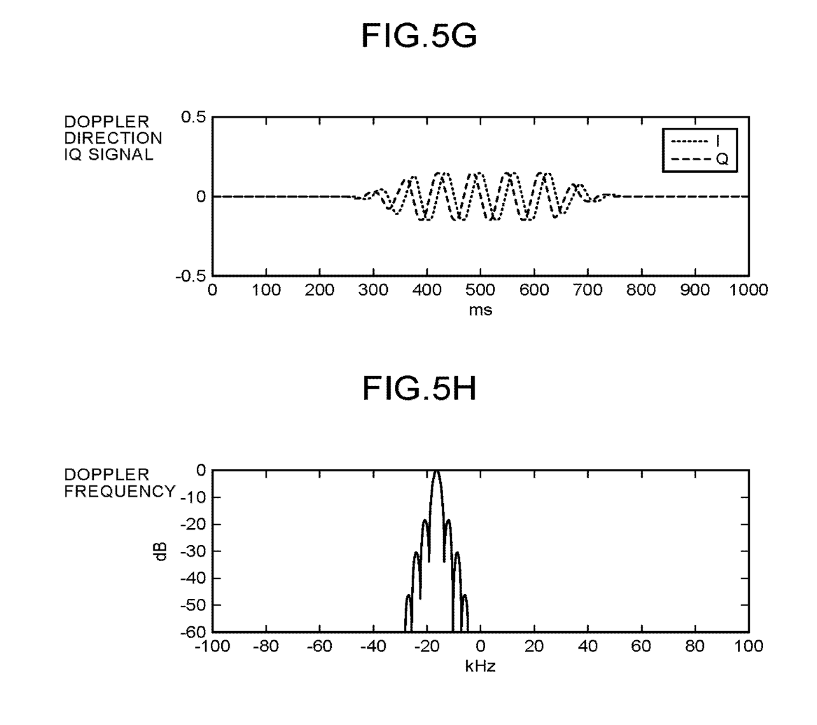

Here, a point reflector is regarded as a source of generation of a clutter. FIGS. 5A to 5H are diagrams that illustrate an example of a simulation performed in a case where a point reflector moves in a direction separating from an ultrasonic beam. FIGS. 5A to 5D illustrate cases where reflected wave signals of all the channels CH are not saturated, and FIGS. 5E to 5H illustrate examples of cases where reflected wave signals of some channels CH are saturated.

FIGS. 5A and 5E illustrate examples of an RF signal in a distance direction. More specifically, FIGS. 5A and 5E illustrate reception signals (RF signals) that are received first to fourth times in a case where ultrasonic waves are transmitted four times. Here, the horizontal axes illustrated in FIGS. 5A and 5E represent the time, in other words, the distance direction, and the vertical axes illustrated in FIGS. 5A and 5E represent the transmission sequence. For example, “1” represented in the vertical axis illustrated in each of FIGS. 5A and 5E represents a reception signal received at the time of first transmission, and “2” represented in the vertical axis illustrated in each of FIGS. 5A and 5E represents a reception signal received at the time of second transmission.

FIGS. 5B and 5F illustrate examples of I signals and Q signals in the distance direction. Here, FIGS. 5B and 5F illustrate IQ signals converted from reception signals, similar to FIGS. 5A and 5E, in a case where an ultrasonic wave is transmitted four times. Here, the horizontal axis illustrated in each of FIGS. 5B and 5F represents the time, in other words, the distance direction, and the vertical axis illustrated in each of FIGS. 5B and 5F represents the transmission sequence. For example, “1” represented in the vertical axis illustrated in each of FIGS. 5B and 5F represents IQ signals acquired from a reception signal received at the time of first transmission, and “2” represented in the vertical axis illustrated in each of FIGS. 5B and 5F represents IQ signals acquired from a reception signal received at the time of second transmission.

FIGS. 5C and 5G illustrate examples of IQ signals in a Doppler direction that are generated from four IQ signals illustrated in FIGS. 5B and 5F. Here, the horizontal axis illustrated in each of FIGS. 5C and 5G represents the time, in other words, the Doppler direction, and the vertical axis illustrated in each of FIGS. 5C and 5G represents the amplitude.

FIGS. 5D and 5H illustrate examples of Doppler shifts calculated using the IQ signals illustrated in FIGS. 5C and 5G. Here, the horizontal axis illustrated in each of FIGS. 5D and 5H represents the Doppler frequency, and the vertical axis illustrated in each of FIGS. 5C and 5G represents decibels. In a case where a point reflector (clutter) moves in a direction separating from an ultrasonic beam, as illustrated in FIG. 5D, also in a case where signals are not saturated and, as illustrated in FIG. 5H, also in a case where signals are saturated, there is no large change in a Doppler spectrum. In addition, also when the Doppler spectrum changes to a degree illustrated in FIG. 5H, a clutter can be suppressed using an MTI filter.

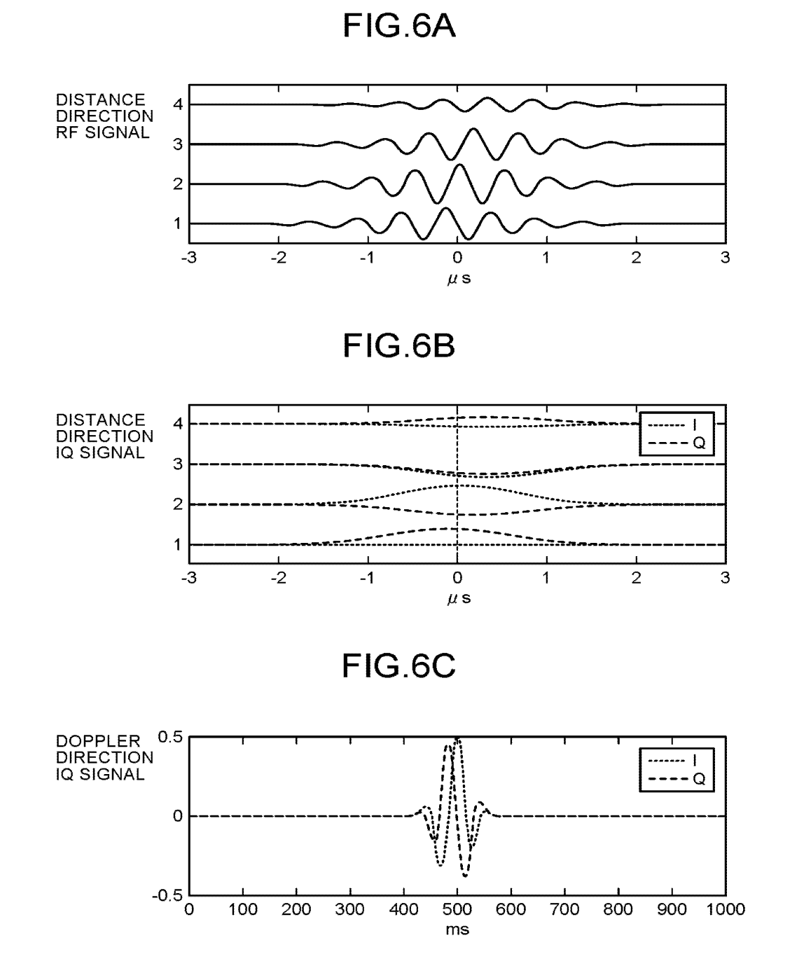

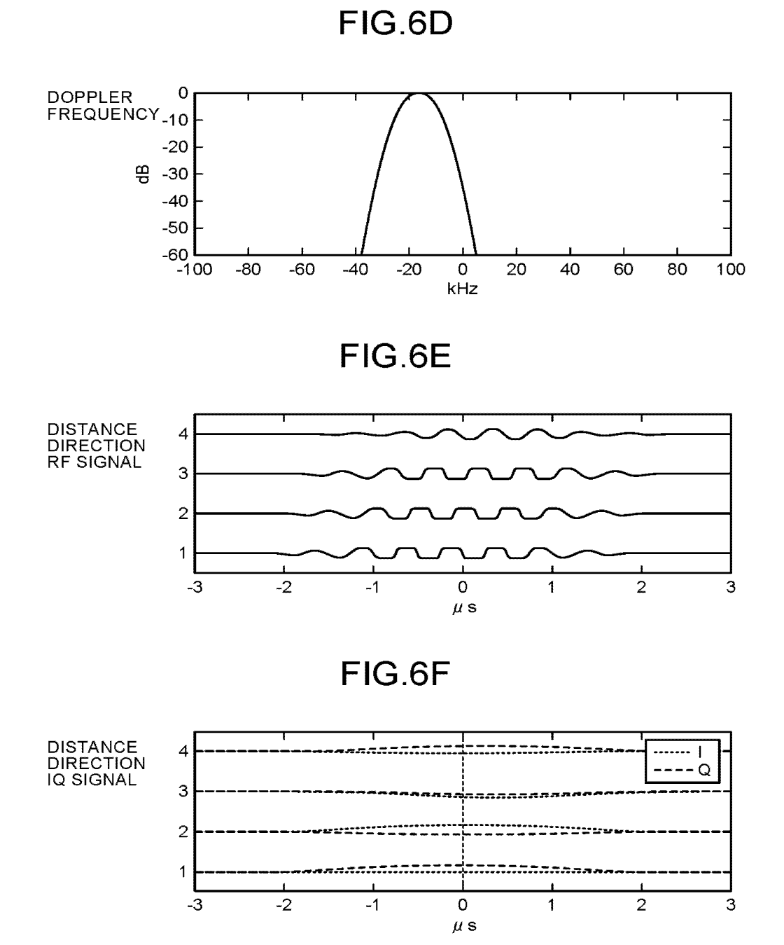

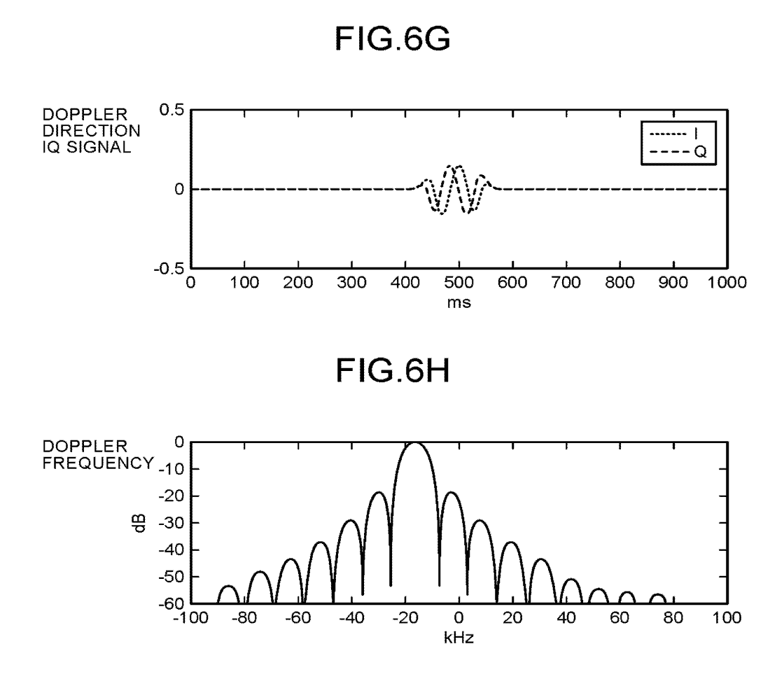

FIGS. 6A to 6H are diagrams that illustrate an example of a simulation performed in a case where a point reflector moves to traverse an ultrasonic beam at a high speed. FIGS. 6A to 6D illustrate cases where reflected wave signals of all the channels CH are not saturated, and FIGS. 6E to 6H illustrate examples of cases where reflected wave signals of some channels CH are saturated. FIGS. 6A and 6E, similar to FIGS. 5A and 5E, illustrate RF signals in the distance direction. FIGS. 6B and 6F, similar to FIGS. 5B and 5F, illustrate I signals and Q signals in the distance direction. FIGS. 6C and 6G, similar to FIGS. 5C and 5G, illustrate IQ signals in the Doppler direction that are generated from four IQ signals.

FIGS. 6D and 6H, similar to FIGS. 5D and 5H, illustrate Doppler shifts. Here, as illustrated in FIG. 6D, in a case where there is no saturation, the Doppler spectrum only expands to some degrees. For this reason, in the case illustrated in FIG. 6D, by setting the cutoff frequency of the MTI filter to be higher than that of the cases illustrated in FIGS. 5D and 5H, a clutter can be suppressed. However, as illustrated in FIG. 6H, in a case where saturation is present, the Doppler spectrum expands up to near a Nyquist frequency, and it is difficult to suppress a clutter using the MTI filter. Differences in the cases illustrated in FIGS. 5D and 5H and FIGS. 6D and 6H are that changes in the envelops are steeper in the cases illustrated in FIGS. 6D and 6H than in the cases illustrated in FIGS. 5D and 5H. In an actual living body, a tissue hardly moves at a high speed in this way. However, in case of a specular reflector, as an angle changes even in a minute displacement, the envelop abruptly changes. In other words, the artifacts illustrated in FIG. 2 are caused as a signal is specularly reflected from a reception element that is a point on the diaphragm and is specularly reflected or not due to a motion of the diaphragm, and a reception signal acquired by the element is saturated at the time of specular reflection and is not saturated at the time of no specular reflection.

Such a problem occurs also in a case other than the “plane wave transmission+all-raster parallel simultaneous reception”. For example, in a case where a specular reflector is moving when eight-direction parallel simultaneous reception is performed, artifacts are observed to be approximately vertical with respect to an ultrasonic beam among eight rasters, and the same problem is checked.

As illustrated in FIG. 2, while the side lobe level is high, the resolution of a blood flow signal in the orientation direction is good. The reason for this is that, originally, a blood flow signal is weak, and imaging is performed only in the range of a main lobe. As a method for enhancing a transmission sound field, while there is a method in which transmission, of which the direction is changed, is performed for a plurality of directions, and coherent composition is performed, there are problems in that the frame rate decreases, and a high-speed flow flow is eliminated by a low path filter (LPF) process. Since an artifact having a circular arc shape is generated only in a specific case, and such an artifact is not generated in the other cases, it is preferable that this problem is solved using a method having no trade-off.

Such an artifact having a circular arc shape is not generated unless a reflected wave signal is saturated in the reception circuitry 112. For this reason, a method for preventing the saturation in the reception circuitry 112 may be considered as a countermeasure. Thus, the ultrasonic diagnostic apparatus 1 according to the first embodiment does not use a saturated channel for beam forming. More specifically, the ultrasonic diagnostic apparatus 1 according to the first embodiment determines whether or not a reflected wave signal received by each channel of the ultrasonic probe 11 is saturated. Then, the ultrasonic diagnostic apparatus 1 according to the first embodiment generates reflected wave data through a phasing addition process using an output signal for which a predetermined process is performed based on a result of the determination.

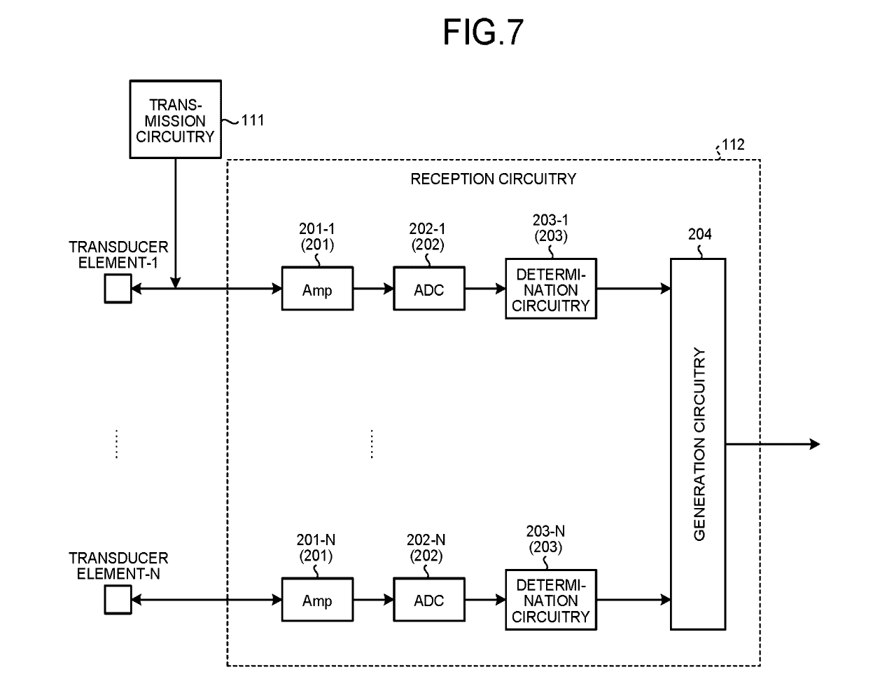

FIG. 7 is a block diagram that illustrates an example of the configuration of the reception circuitry 112 according to the first embodiment. As illustrated in FIG. 7, the reception circuitry 112 is connected to N transducer elements (Transducer element-1, . . . , Transducer element-N). Here, each of the transducer elements corresponds to each channel.

As illustrated in FIG. 7, the reception circuitry 112 includes: amplification circuitry 201-1; an A/D converter 202-1; and determination circuitry 203-1 as sub circuits used for processing a reflected wave signal received by Transducer element-1. Similarly, the reception circuitry 112 includes: amplification circuitry 201-N; an A/D converter 202-N; and determination circuitry 203-N as sub circuits used for processing a reflected wave signal received by Transducer element-N. Here, in a case where the amplification circuitry 201-1 and the amplification circuitry 201-N do not need to be discriminated from each other, the amplification circuitry will be described as amplification circuitry 201. In addition, in a case where the A/D converter 202-1 and the A/D converter 202-N do not need to be discriminated from each other, the A/D converter will be described as an A/D converter 202, and, in a case where the determination circuitry 203-1 and the determination circuitry 203-N do not need to be discriminated from each other, the determination circuitry will be described as determination circuitry 203. In other words, in the reception circuitry 112, the amplification circuitry 201, the A/D converter 202, and the determination circuitry 203 are disposed for each transducer element (channel). As described above, the amplification circuitry 201 performs a gain correction process by amplifying a reflected wave signal for each channel. In addition, the A/D converter 202 performs an A/D conversion of the gain-corrected reflected wave signal.

The determination circuitry 203 determines whether or not a reflected wave signal received by each channel of the ultrasonic probe 11 is saturated. Here, the determination circuitry 203 determines saturation by using that an output value of the A/D converter 202 is a positive upper limit or a negative lower limit as a digital value. Then, the determination circuitry 203 outputs a result of the determination to generation circuitry 204. In addition, in a case where the value of a reflected wave signal is a predetermined threshold or more, the determination circuitry 203 may determine that the reflected wave signal is saturated.

The generation circuitry 204 generates reflected wave data by performing a phasing addition process using an output signal for which a predetermined process corresponding to a result of the determination acquired by the determination circuitry 203 has been performed. For example, the generation circuitry 204 sets the depth data of the channel in which the saturation has been detected to “O”. In other words, the generation circuitry 204 does not use the saturated channel for beam forming. Here, instead of setting the data to “0”, by multiplying the data by a coefficient determined in advance in the range of 0 to 1, the generation circuitry 204 may decrease a contribution thereof. In other words, the generation circuitry 204 generates reflected wave data by using an output value acquired by multiplying the reflected wave signal of the saturated channel by a predetermined coefficient of one or less as an output signal. The generation circuitry 204 outputs the generated reflected wave data to the Doppler processing circuitry 130.

The Doppler processing circuitry 130 generates Doppler data acquired by extracting motion information based on the Doppler effect of a moving body as each sample point within the scanning area by using the reflected wave data received from the reception circuitry 112. Then, the Doppler processing circuitry 130 transmits the generated Doppler data to the image generating circuitry 140. In this way, the image generating circuitry 140 generates ultrasonic wave image data based on the data generated by the Doppler processing circuitry 130.

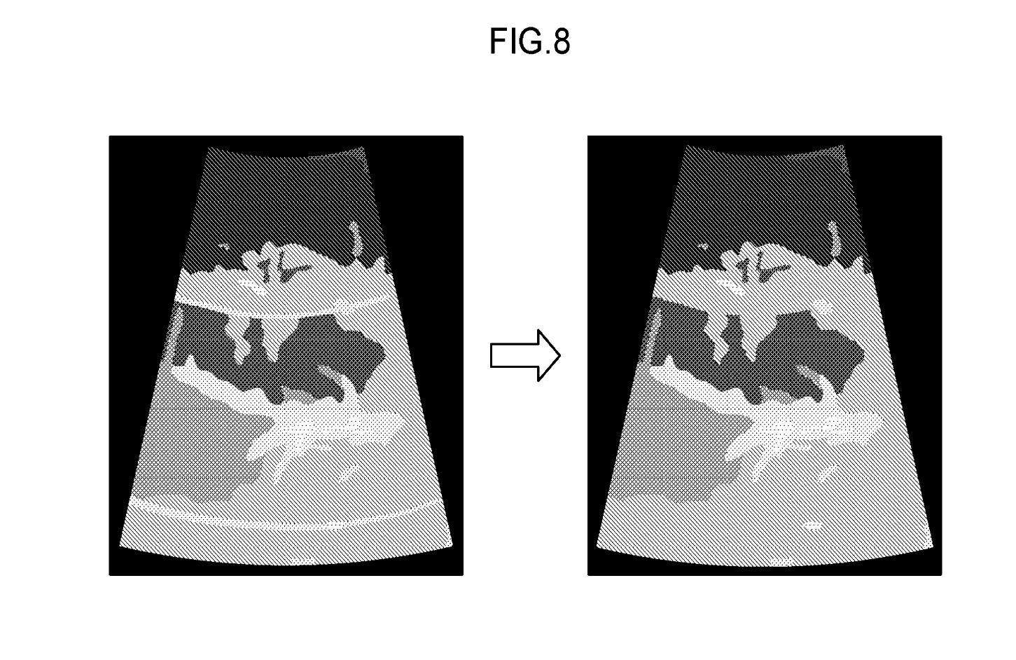

As described above, the ultrasonic diagnostic apparatus 1 according to the first embodiment does not use a saturated channel for beam forming or uses a saturated channel for beam forming with the influence of the saturated channel decreased. As a result, according to the first embodiment, an artifact according to a strong reflector can be decreased. FIG. 8 is a diagram that illustrates an effect of the ultrasonic diagnostic apparatus 1 according to the first embodiment.

FIG. 8 illustrates an example of a case where blood flow information is power-displayed. A left diagram in FIG. 8, similar to FIG. 2, illustrates a case where artifacts are generated in a circular arc shape including a strong reflector in a case where power display of a blood flow is performed using “plane wave transmission+all-raster parallel simultaneous reception” according to the conventional technology. On the other hand, a right diagram in FIG. 8, illustrates a case where power display of a blood flow is performed using “plane wave transmission+all-raster parallel simultaneous reception” in the ultrasonic diagnostic apparatus 1 according to the first embodiment. As illustrated in the right diagram in FIG. 8, the artifacts having a circular arc shape generated in the left diagram in FIG. 8 disappear. Particularly, in a case where “plane wave transmission+all-raster parallel simultaneous reception” is performed, a remarkable effect is acquired.

According to the first embodiment, a process is performed in which a signal from a specular reflector is suppressed, or the blood flow information of that portion is not displayed. Here, there are cases where the signals of all the channels CH are not used. According to such a process, while there may be a problem in that a structure such as a vessel wall is not displayed on a B-mode image, a blood flow is not present in such a place in a blood flow image, and thus, it does not matter. In addition, the first embodiment can be realized using a relatively simple circuit configuration.

### Second Embodiment

The whole configuration of an ultrasonic diagnostic apparatus 1a according to a second embodiment is the same as the whole configuration of the ultrasonic diagnostic apparatus 1 according to the first embodiment illustrated in FIG. 1 except for a part of the configuration of reception circuitry, and thus, the description thereof will not be presented here. In the second embodiment, processing circuitry 170 causes an ultrasonic probe 11 to perform ultrasonic wave scanning using a data row between frames as a Doppler data row. For example, the processing circuitry 170 causes the ultrasonic probe 11 to perform first ultrasonic wave scanning acquiring information relating to a motion of a moving body within a first scanning range and causes the ultrasonic probe 11 to perform ultrasonic wave scanning of each of a plurality of divided ranges acquired by dividing a second scanning range as second ultrasonic wave scanning acquiring information of the shape of a tissue within the second scanning range in a time divisional manner during the first ultrasonic wave scanning.

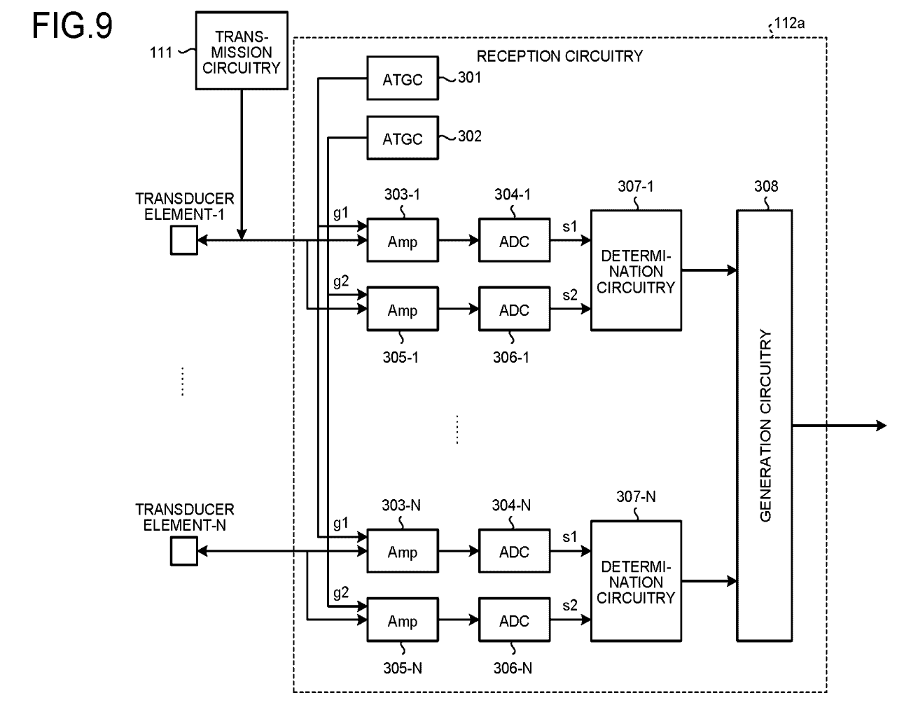

FIG. 9 is a block diagram that illustrates an example of the configuration of reception circuitry 112a according the second embodiment. As illustrated in FIG. 9, the reception circuitry 112a is connected to N transducer elements (Transducer element-1, . . . , Transducer element-N). Here, each of the transducer elements corresponds to each channel.

As illustrated in FIG. 9, the reception circuitry 112a includes: amplification circuitry 303-1; an A/D converter 304-1; amplification circuitry 305-1; an A/D converter 306-1; and determination circuitry 307-1 for processing a reflected wave signal received by Transducer element-1. Here, the amplification circuitry 303-1 and the A/D converter 304-1 configure a first processing system of Transducer element-1, and the amplification circuitry 305-1 and the A/D converter 306-1 configure a second processing system of Transducer element-1. Similarly, the reception circuitry 112a includes: amplification circuitry 303-N; an A/D converter 304-N; amplification circuitry 305-N; an A/D converter 306-N; and determination circuitry 307-N for processing a reflected wave signal received by Transducer element-N. Here, the amplification circuitry 303-N and the A/D converter 304-N configure a first processing system of Transducer element-N, and the amplification circuitry 305-N and the A/D converter 306-N configure a second processing system of Transducer element-N.

Here, in a case where the amplification circuitry 303-1 and the amplification circuitry 303-N do not need to be discriminated from each other, the amplification circuitry will be described as an amplification circuitry 303, and, in a case where the A/D converter 304-1 and the A/D converter 304-N do not need to be discriminated from each other, the A/D converter will be described as an A/D converter 304. In addition, in a case where the amplification circuitry 305-1 and the amplification circuitry 305-N do not need to be discriminated from each other, the amplification circuitry will be described as an amplification circuitry 305, in a case where the A/D converter 306-1 and the A/D converter 306-N do not need to be discriminated from each other, the A/D converter will be described as an A/D converter 306, and, in a case where the determination circuitry 307-1 and the determination circuitry 307-N do not need to be discriminated from each other, the determination circuitry will be described as determination circuitry 307. In other words, in the reception circuitry 112a, the amplification circuitry 303, the A/D converter 304, the amplification circuitry 305, the A/D converter 306, and the determination circuitry 307 are disposed for each transducer element (channel). For a reflected wave signal from one transducer element, two amplification circuits including the amplification circuitry 303 and the amplification circuitry 305 are connected.

In addition, the reception circuitry 112a includes an ATGC 301 and an ATGC 302. The ATGC 301 stores a first gain value, and the ATGC 302 stores a second gain value. The amplification circuitry 303 has a first gain value g1 (for example, g1=100 (40 dB)) that is high, similar to a conventional blood flow imaging method, and performs a gain correction process by amplifying a reflected wave signal for each channel. The amplification circuitry 303 outputs the reflected wave signal for which the gain correction process has been performed to the A/D converter 304. In other words, the amplification circuitry 303 corrects the reflected wave signal of each channel using the first gain value g1, thereby acquiring a first corrected signal from the reflected wave signal.

The amplification circuitry 305 has a second gain value g2 (for example, g2=10 (20 dB)) that is lower than the first gain value g1 and performs a gain correction process by amplifying a reflected wave signal for each channel. The amplification circuitry 305 outputs the reflected wave signal for which the gain correction process has been performed to the A/D converter 306. In other words, the amplification circuitry 305 corrects the reflected wave signal of each channel using the second gain value having a gain value smaller than the first gain value, thereby acquiring a second corrected signal from the reflected wave signal.

The A/D converter 304 performs an A/D conversion of the reflected wave signal of which the gain has been corrected and outputs the reflected wave signal after the A/D conversion to the determination circuitry 307. In addition, the A/D converter 306 performs an A/D conversion of the reflected wave signal of which the gain has been corrected and outputs the reflected wave signal after the A/D conversion to the determination circuitry 307.

The determination circuitry 307 determines whether or not a reflected wave signal received by each channel of the ultrasonic probe 11 is saturated through at least first ultrasonic wave scanning. Here, the determination circuitry 307 determines whether or not the reflected wave signal of each channel is saturated by comparing the first corrected signal with the second corrected signal. For example, in a case where the absolute value of a difference between the first corrected signal and the second corrected signal is a predetermined threshold or less, the determination circuitry 307 determines that the reflected wave signal is not saturated. Here, in a case where no saturation is present, when an output signal s1 of the A/D converter 304 passing through the amplification circuitry 303 is 1/10, a signal close to an output signal s2 of the A/D converter 306 passing through the amplification circuitry 305 is acquired. Based on such a situation, in a case where the absolute value of the difference between both the signals is less than the threshold Th, the determination circuitry 307 determines no saturation. On the other hand, in a case where the absolute value of the difference is more than the threshold Th, the determination circuitry 307 determines that saturation is present.

Generation circuitry 308 generates reflected wave data by using the first corrected signal as an output signal in a case where the reflected wave signal is determined not to be saturated or by using a signal acquired by mixing the first corrected signal and the second corrected signal at a predetermined ratio as an output signal in a case where the reflected wave signal is determined to be saturated.

Hereinafter, details of the process performed by the generation circuitry 308 will be described.

The generation circuitry 308 can appropriately use three kinds of determination processes including Determination System 1 to Determination System 3 represented below. First, in Determination System 1, the determination circuitry 307 outputs s=s1 when s2−s1*g2/g1<Th (a case where s2−s1*g2/g1<0 due to noises is also included herein). On the other hand, the generation circuitry 308 outputs s=0 when s2−s1*g2/g1>=Th.

In Determination System 2, the generation circuitry 308 switches the threshold between s1 and s2. More specifically, the determination circuitry 307 outputs s=s1 when s2−s1*g2/g1<Th. On the other hand, the generation circuitry 308 outputs s=s2*g1/g2 when s2−s1*g2/g1>=Th.

In Determination System 3, the generation circuitry 308 outputs s1 and s2 in a mixed manner. More specifically, the generation circuitry 308 outputs s=s1 when s2−s1*g2/g1<Th. On the other hand, the generation circuitry 308 outputs s=a*s1+(1−a)*s2*g1/g2 when s2−s1*g2/g1>=Th. Here, a is a function of s1 and s2 and takes a value in the range of 0 to 1. For example, by using Th2 as the threshold, when s2−s1*g2/g1−Th<Th2, a=(s2−s1*g2/g1−Th)/Th2, and, when s2−s1*g2/g1−Th>=Th2, a=1(s=s1). Here, the signals s1, s2, and s are calculated at the output rates of the A/D converter 304 and the A/D converter 306.

In this way, the generation circuitry 308 mixes the first corrected signal and the second corrected signal at a predetermined ratio by appropriately using three kinds of determination processes including Determination System 1 to Determination System 3, thereby generating reflected wave data. The generation circuitry 308 outputs the generated reflected wave data to the Doppler processing circuitry 130.

The Doppler processing circuitry 130 generates Doppler data acquired by extracting motion information based on the Doppler effect of a moving body as each sample point within the scanning area by using the reflected wave data received from the reception circuitry 112. Then, the Doppler processing circuitry 130 transmits the generated Doppler data to the image generating circuitry 140. In this way, the image generating circuitry 140 generates ultrasonic wave image data based on the data generated by the Doppler processing circuitry 130.

As described above, the ultrasonic diagnostic apparatus 1a according to the second embodiment does not use a saturated channel for beam forming or uses a saturated channel for beam forming with the influence of the saturated channel decreased. As a result, according to the second embodiment, an artifact according to a strong reflector can be decreased.

First Modified Example of Second Embodiment

While a case of the configuration in which two amplification circuits and two A/D converters are constantly connected for one channel has been illustrated in FIG. 9, the embodiment is not limited thereto. For example, as the first modified example of the second embodiment, a configuration may be employed in which a switch such as switching circuitry is further included, and a reflected wave signal from a transducer element and amplification circuitry and subsequent circuits can be freely connected by using the switch. For example, the state may be switched according to the number of channels used for ultrasonic wave scanning between a first state in which the ultrasonic probe 11 is connected to the amplification circuitry 303 and the amplification circuitry 305 and a second state in which the ultrasonic probe 11 is connected to one of the amplification circuitry 303 and the amplification circuitry 305. Then, in a case where a plurality of channels are used as in three dimensional scanning, one amplification circuitry and subsequent circuitry are connected for one channel, and, in case of the two-dimensional blood flow imaging method, two amplification circuitry and subsequent circuitry are connected for one channel. The switching circuitry, for example, performs switching according to the number of channels used for ultrasonic wave scanning between the first state and the second state under the control of the processing circuitry 170.

According to the first modified example of the second embodiment, since the number of channels CH, which are of a degree unnecessary at the time of performing two-dimensional scanning, are included in a system performing three-dimensional ultrasonic wave scanning in real time, by effectively using the remaining circuitry, the cost problem can be solved.

Second Modified Example of Second Embodiment

As a second modified example of the second embodiment, a B mode image may be generated using a signal having a lower gain. When a B mode image is generated based on an ultrasonic wave signal that is transmitted or received for normal blood flow information, the gain is high. Accordingly, there are many cases where a signal received from a strong reflector is saturated, and therefore, a tissue image cannot be displayed at good contrast. However, by using a signal of which gain has been lowered, a B mode image having good contrast can be generated. In this way, according to an ultrasonic diagnostic apparatus of a second modified example of the second embodiment, scanning for a B mode does not need to be performed separately from scanning for the Doppler mode, and accordingly, the frame rate can be raised. In addition, by completely excluding the influence of remaining multiplexing according to the B mode scanning, a blood flow image having a low noise level can be displayed. In such a case, the generation circuitry 308 generates reflected wave data by using the second corrected signal acquired through a correction using the second gain value that is less than the first gain value as an output signal. Then, the generation circuitry 308 outputs the generated reflected wave data to the B-mode processing circuitry 120.

The B-mode processing circuitry 120 generates B mode data by using the reflected wave data received from the reception circuitry 112. Then, the B-mode processing circuitry 120 transmits the generated B mode data to the image generating circuitry 140. In this way, the image generating circuitry 140 generates ultrasonic wave image data based on the data generated by the B-mode processing circuitry 120.

In the second embodiment, while the configuration for beam forming using hardware has been described, the embodiment is not limited thereto. For example, a configuration for beam forming by transmitting the output data of the A/D converter to a memory and executing software using the CPU may be employed.

### Third Embodiment

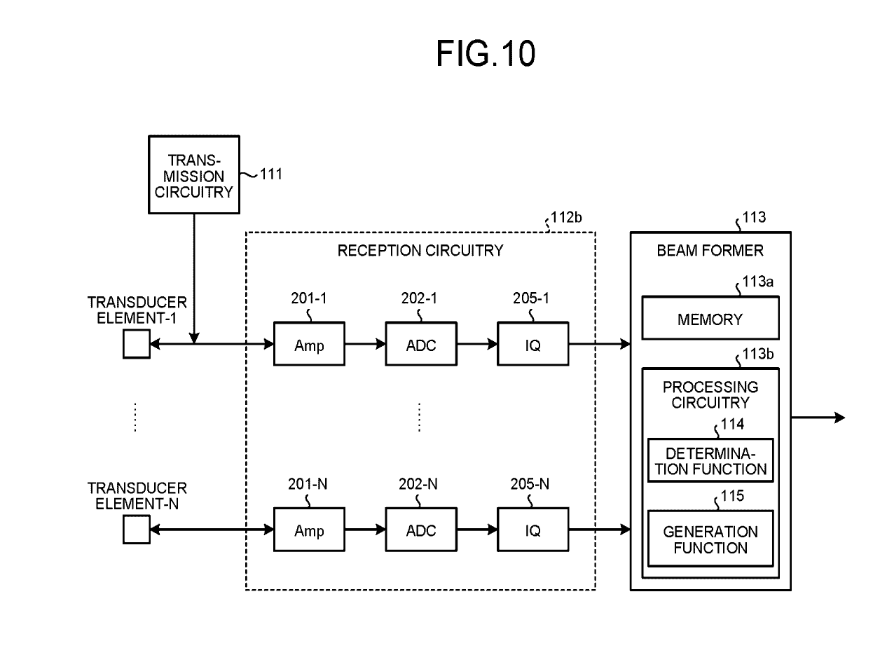

The whole configuration of an ultrasonic diagnostic apparatus 1b according to a third embodiment is the same as the configuration example illustrated in FIG. 1 except that some of the functions of reception circuitry are different, and a beam former is arranged on a later stage of the reception circuitry and on a former stage of B-mode processing circuitry and Doppler processing circuitry. For this reason, in the third embodiment, only the reception circuitry and the beam former will be described with reference to FIG. 10. FIG. 10 is a diagram that illustrates an example of reception circuitry 112b and a beam former 113 according to the third embodiment.

As illustrated in FIG. 10, the reception circuitry 112b is connected to N transducer elements (Transducer element-1, . . . , Transducer element-N). Here, each of the transducer elements corresponds to each channel. As illustrated in FIG. 10, the reception circuitry 112b includes: amplification circuitry 201-1; an A/D converter 202-1; and quadrature detection circuitry (referred to as “IQ” as is appropriate) 205-1 for processing a reflected wave signal received by Transducer element-1. Similarly, the reception circuitry 112b includes: amplification circuitry 201-N; an A/D converter 202-N; and quadrature detection circuitry 205-N for processing a reflected wave signal received by Transducer element-N.

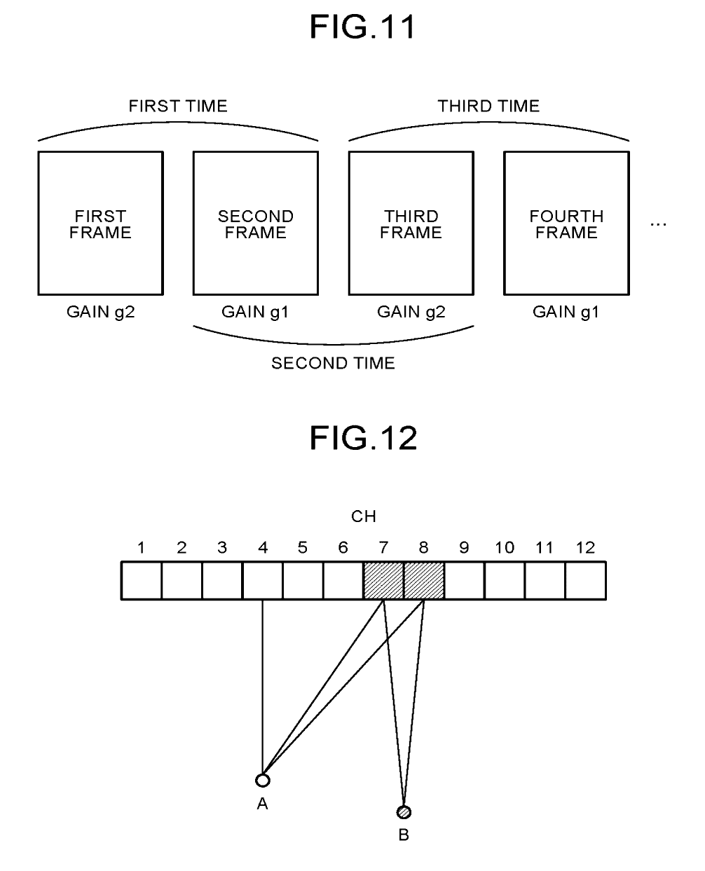

When the gain of a received reflected wave signal is corrected in units of frames, the amplification circuitry 201 alternately corrects a gain value to a first gain value and a second gain value less than the first gain value for every adjacent frame. Then, the amplification circuitry 201 acquires a first corrected signal acquired by correcting a reflected wave signal in units of frames using the first gain value and a second corrected signal acquired by correcting a reflected wave signal using the second gain value. In other words, the amplification circuitry 201 changes the gain value for every frame. FIG. 11 is a diagram that illustrates the third embodiment.

As illustrated in FIG. 11, for example, the amplification circuitry 201 sets a higher gain (first gain value) g1 (for example, g1=100 (40 dB)) to an even-numbered frame and a lower gain (second gain value) g2 (for example, g2=10 (20 dB)) to an odd-numbered frame for every two frames. For example, the amplification circuitry 201 sets the gain g2 to a first frame and sets the gain g1 to a second frame. In this way, a second corrected signal is acquired from the first frame, and a first corrected signal is acquired from the second frame. In addition, the amplification circuitry 201 sets the gain g2 to a third frame and sets the gain g1 to a fourth frame. In this way, a second corrected signal is acquired from the third frame, and a first corrected signal is acquired from the fourth frame.

The A/D converter 202 performs an A/D conversion of the gain-corrected reflected wave signal and outputs the reflected wave signal after the A/D conversion to quadrature detection circuitry 205. Then, the quadrature detection circuitry 205 transmits the output of each channel after the A/D conversion to the beam former 113.

The beam former 113 performs beam forming using the output of each channel after the A/D conversion. The beam former 113 may be realized either by hardware or by software. In the third embodiment, a case will be described in which the beam former 113 is realized by software.

The beam former 113 includes a memory 113a and processing circuitry 113b. The memory 113a maintains a capacity for storing reflected wave signals of two frames corresponding to two types of gains.

The processing circuitry 113b performs a determination function 114 and a generation function 115. Here, for example, processing functions performed by the determination function 114 and the generation function 115 that are constituent elements of the processing circuitry 113b illustrated in FIG. 10 are recorded in the storage circuitry 160 in the form of a computer-executable program. The processing circuitry 113b is a processing realizing a function corresponding to each program by reading the program from the storage circuitry 160 and executing the program. In other words, the processing circuitry 113b that is in a state in which each program is read has each function illustrated inside the processing circuitry 113b illustrated in FIG. 10.

The determination function 114 is a function similar to that of the determination circuitry 307 illustrated in FIG. 9. In other words, the determination function 114 determines whether or not a reflected wave signal in unit of frames is saturated by comparing the first corrected signal with the second corrected signal. The generation function 115 is a function similar to that of the generation circuitry 308 illustrated in FIG. 9. In other words, the generation function 115 generates reflected wave data by using the first corrected signal as an output signal in a case where the reflected wave signal is determined not to be saturated or by using a signal acquired by mixing the first corrected signal and the second corrected signal at a predetermined ratio as an output signal in a case where the reflected wave signal is determined to be saturated. The generation function 115 outputs the generated reflected wave data to the Doppler processing circuitry 130.

The Doppler processing circuitry 130 generates Doppler data acquired by extracting motion information based on the Doppler effect of a moving body as each sample point within the scanning area by using the reflected wave data received from the beam former 113. Then, the Doppler processing circuitry 130 transmits the generated Doppler data to the image generating circuitry 140. In this way, the image generating circuitry 140 generates ultrasonic wave image data based on the data generated by the Doppler processing circuitry 130.

Modified Example of Third Embodiment

As a modified example of the third embodiment, a B mode image may be generated by using a signal having a lower gain. For example, the image generating circuitry 140 generates an image including information of a tissue shape by using the second gain value. In such a case, the processing circuitry 170 causes the ultrasonic probe 11 to perform ultrasonic wave scanning acquiring information relating to a motion of a moving body. In this way, scanning for a B mode does not need to be performed, and accordingly, the frame rate can be raised, and the influence of remaining multiplexing according to the B mode scanning can be completely excluded. In the third embodiment and the modified examples of the third embodiment, an optimal method is “plane wave transmission+all-raster parallel simultaneous reception”. In this way, the influence of the motion can be reduced.

### Fourth Embodiment

In the first embodiment, a case has been illustrated in which a saturated channel is not used for the beam forming or is used for the beam forming with the influence of the saturated channel decreased. However, as illustrated in FIG. 5H, in a case where a point reflector (clutter) moves in a direction separating from an ultrasonic wave beam, even when a signal of a certain channel CH is saturated, the clutter can be suppressed using an MTI filter. In other words, in a case where specular reflection is not performed, a clutter can be suppressed using the MTI filter. In such a case, the beam forming may be performed using a saturated signal. Thus, in a fourth embodiment, a case will be described in which it is determined whether or not specular reflection is performed, and, in a case where specular reflection is not performed, a saturated signal is also used for the beam forming.

The whole configuration of an ultrasonic diagnostic apparatus according to the fourth embodiment is the same as the configuration example illustrated in FIG. 1 except that some of the functions of reception circuitry are different, and a beam former is arranged on a later stage of the reception circuitry and on a former stage of B-mode processing circuitry and Doppler processing circuitry. In addition, the configuration of the reception circuitry and the beam former according to the fourth embodiment is the same as the configuration example illustrated in FIG. 10 except that a determination function 114 and some of the functions of the generation function 115 are different.

In the fourth embodiment, processing circuitry 170 causes an ultrasonic probe 11 to perform ultrasonic wave scanning using a data row between frames as a Doppler data row (see Japanese Patent No. 3724846 and Japanese Patent Application Publication No. 2014-42823). For example, the processing circuitry 170 causes the ultrasonic probe 11 to perform first ultrasonic wave scanning acquiring information relating to a motion of a moving body within a first scanning range and causes the ultrasonic probe 11 to perform ultrasonic wave scanning of each of a plurality of divided ranges acquired by dividing a second scanning range as second ultrasonic wave scanning acquiring information of the shape of a tissue within the second scanning range in a time divisional manner during the first ultrasonic wave scanning. Amplification circuitry 201 according to the fourth embodiment, different from the amplification circuitry 201 according to the third embodiment, does not perform the changing of the gain for each frame. In other words, the amplification circuitry 201 according to the fourth embodiment, for example, like the first gain value g1 according to the second embodiment, uses a fixed high gain value as in a conventional case.

The determination function 114 has a function similar to that of the determination circuitry 203 illustrated in FIG. 7. In other words, the determination function 114 determines whether or not a reflected wave signal received by each channel of the ultrasonic probe 11 is saturated. Then, the determination circuitry 203 outputs a result of the determination to the generation circuitry 204.

The generation function 115 determines whether or not specular reflection is performed based on a result of the determination of the determination function 114 and generates reflected wave data based on a result of the determination. The processing operation of the generation function 115 according to the fourth embodiment will be described with reference to FIG. 12. FIG. 12 is a diagram that illustrates the processing operation of the generation function 115 according to the fourth embodiment.

As illustrated in FIG. 12, a case will be described in which a specular reflector B is present when beam forming at the position of a point A is performed. Here, an echo from the specular reflector B has characteristics that only an ultrasonic beam that is incident to and reflected from the specular reflector B at right angles causes specular reflection. For this reason, as illustrated in FIG. 12, a channel CH-7 positioned in a vertical direction in the incidence direction of an ultrasonic beam with respect to the specular reflector B and a plurality of channels CH adjacent to this channel CH-7 are saturated. FIG. 12 illustrates a case where channels CH-7 and CH-8 are saturated. On the other hand, channels CH other than the channels CH-7 and CH-8 are not saturated according to the influence of the specular reflector B.

In a conventional system, in a case where a reception signal received from each channel CH is an RF signal, a time delay is applied to the reception signal received from each channel CH, in case of IQ signals, a time delay and a phase delay are applied to a reception signal received from each channel CH, and an addition process is performed for all the channels CH. On the other hand, according to the fourth embodiment, in a case where N or more channels CH acquired by combining a channel CH (referred to as a saturated channel CH) detected to be saturated by the determination function 114 and channels CH (adjacent channels CH) adjacent to the saturated channel CH are saturated, the generation function 115 decreases the influences of the saturated channel CH and the adjacent channels CH so as to be used for beam forming. In other words, the generation function 115 does not use the saturated channel CH and the adjacent channels CH for beam forming. Alternatively, the generation function 115 performs beam forming by multiplying reflected wave signals of the saturated channel CH and the adjacent channels CH by a coefficient of one or less that is determined in advance. Here, N is a value determined according to a position in the depth direction at which the specular reflector is located and is set to be small at a short distance and is set to be large at a long distance.

In the example illustrated in FIG. 12, in a case where N=2, the generation function 115 determines that signals of the channels CH7 and CH8 to be saturated according to echoes from the specular reflector B and does not use the reflected wave signals of the channels CH7 and CH8 for beam forming. On the other hand, for example, even in a case where a channel CH-2 is saturated, when a channel CH-1 or CH-3 adjacent to the channel CH-2 is not saturated, the generation function 115 does not determine that the channels are saturated according to echoes from the specular reflector B but uses the reflected wave signal of the channel CH-2 for beam forming. In other words, as a result of the ultrasonic wave scanning, in a case where reflected wave signals of a plurality of adjacent channels corresponding to a predetermined number or more among reflected wave signals received from the channels are saturated, the generation function 115 generates reflected wave data by using an output value acquired by multiplying the reflected wave signals of a plurality of channels by a predetermined coefficient of one or less as an output signal. Then, the generation function 115 outputs the generated reflected wave data to the Doppler processing circuitry 130.

The Doppler processing circuitry 130 generates Doppler data acquired by extracting motion information based on the Doppler effect of a moving body as each sample point within the scanning area by using the reflected wave data received from the beam former 113. Then, the Doppler processing circuitry 130 transmits the generated Doppler data to the image generating circuitry 140. In this way, the image generating circuitry 140 generates ultrasonic wave image data based on the data generated by the Doppler processing circuitry 130.

As described above, according to the fourth embodiment, an artifact can be suppressed by suppressing only signals received from a specular reflector without setting a plurality of gains. For example, as illustrated in FIG. 5H, in a case where, although the channel CH is saturated, specular reflection is not performed, and a clutter can be suppressed by using an MTI filter, beam forming can be performed using the saturated signal. In the fourth embodiment, while a configuration for performing beam forming using software has been described, the configuration may be realized by hardware.

### Fifth Embodiment

In imaging blood flow information, the Doppler processing circuitry performs a moving target indicator (MTI) filter process using reflected wave signals after the beam forming. Here, in a case where saturated data is present inside a packet, the data is discontinuous. For this reason, in an MTI filter that is a high pass filter (HPF), a signal passes through a discontinuous point, and a signal originated from a tissue is incorrectly recognized as a blood flow signal. For example, in a conventional system, there are cases where, as a specular reflector rotates according to a minute motion, a signal originated from a tissue passes through the MTI filter. In such cases, a change in the envelope becomes steep, and, even in a case where there is no saturation, as illustrated in FIG. 6D, the Doppler spectrum expands. In a case where there is saturation, as illustrated in FIG. 6H, the Doppler spectrum expands up to near a Nyquist frequency. In such a case, a signal originated from a tissue passes through a general MTI filter. As a result, the signal originated from the tissue is displayed as a blood flow signal. In this way, after the beam forming, it cannot be detected whether or not a reflected wave signal is saturated.

Bases on such a situation, in a fifth embodiment, it is determined whether or not a signal is saturated in each channel CH, and, in a case where a saturated signal is included, data of the channel CH at that time is not used for beam forming.

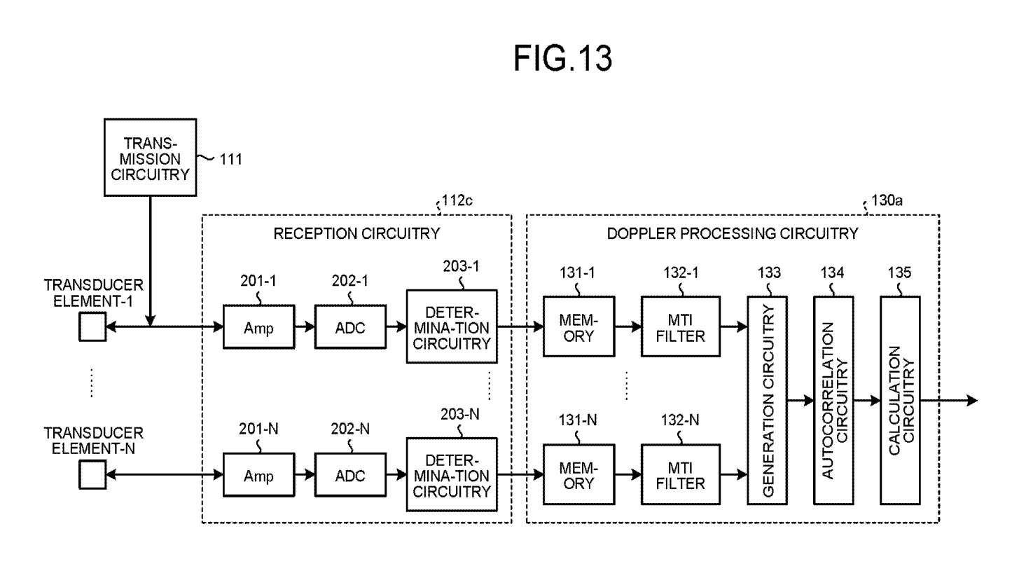

The whole configuration of an ultrasonic diagnostic apparatus according to the fifth embodiment is the same as the configuration example illustrated in FIG. 1 except that parts of the configurations and the functions of reception circuitry and Doppler processing circuitry are different. For this reason, in the fifth embodiment, only the reception circuitry and the Doppler processing circuitry will be described with reference to FIG. 13. FIG. 13 is a diagram that illustrates an example of reception circuitry 112c and Doppler processing circuitry 130a according to the fifth embodiment. In the fifth embodiment, processing circuitry 170 causes an ultrasonic probe 11 to perform ultrasonic wave scanning using a data row between frames as a Doppler data row (see Japanese Patent No. 3724846 and Japanese Patent Application Publication No. 2014-42823).

As illustrated in FIG. 13, the reception circuitry 112c according to the fifth embodiment is connected to N transducer elements (Transducer element-1, . . . , Transducer element-N). Here, each of the transducer elements corresponds to each channel. The reception circuitry 112c includes: amplification circuitry 201-1; an A/D converter 202-1; and determination circuitry 203-1 as sub circuits used for processing a reflected wave signal received by Transducer element-1. Similarly, the reception circuitry 112 includes: amplification circuitry 201-N; an A/D converter 202-N; and determination circuitry 203-N as sub circuits used for processing a reflected wave signal received by Transducer element-N. Here, in a case where the amplification circuitry 201-1 and the amplification circuitry 201-N do not need to be discriminated from each other, the amplification circuitry will be described as amplification circuitry 201. In addition, in a case where the A/D converter 202-1 and the A/D converter 202-N do not need to be discriminated from each other, the A/D converter will be described as an A/D converter 202, and, in a case where the determination circuitry 203-1 and the determination circuitry 203-N do not need to be discriminated from each other, the determination circuitry will be described as determination circuitry 203. As described above, the amplification circuitry 201 performs a gain correction process by amplifying a reflected wave signal for each channel. In addition, the A/D converter 202 performs an A/D conversion of the gain-corrected reflected wave signal.

The determination circuitry 203 determines whether or not a reflected wave signal received by each channel of the ultrasonic probe 11 is saturated. Then, the determination circuitry 203 outputs a result of the determination to the Doppler processing circuitry 130a.

The Doppler processing circuitry 130a includes a memory 131-1 and an MTI filter 132-1 as sub circuits performing a process based on a result of the determination acquired by the determination circuitry 203-1 for a reflected wave signal received by Transducer element-1. In addition, the Doppler processing circuitry 130a includes a memory 131-N and an MTI filter 132-N as sub circuits performing a process based on a result of the determination acquired by the determination circuitry 203-N for a reflected wave signal received by Transducer element-N. Here, in a case where the memory 131-1 and the memory 131-N do not need to be discriminated from each other, the memory will be described as a memory 131, and, in a case where the MTI filter 132-1 and the MTI filter 132-N do not need to be discriminated from each other, the MTI filter will be described as an MTI filter 132. The Doppler processing circuitry 130a includes: generation circuitry 133; autocorrelation circuitry 134; and calculation circuitry 135.

The memory 131 stores reflected wave signals of an ultrasonic wave transmitted a plurality of times in a same scanning line and a result of the determination acquired by the determination circuitry 203 for each reflected wave signal in association with the reflected wave signal. Here, the memory 131 has a capacity capable of storing reflected wave signals of an ultrasonic wave transmitted a plurality of times in the same scanning line.

The MTI filter 132 performs a filter process for reflected wave signals of an ultrasonic wave transmitted a plurality of times in the same scanning line. Here, the MTI filter 132 sets the MTI filter output to “0” in a case where at least one piece of saturated data among data disposed inside L packets is saturated. In a case where there is no saturation in all the data disposed inside the L packets, the MTI filter 132 applies a general MTI filter (for example, a Butterworth infinite impulse response (IIR) filter, a polynomial regression filter, or the like). The MTI filter 132 outputs a result of the process to the generation circuitry 133. Then, the generation circuitry 133 generates reflected wave data through a phasing addition process using the reflected wave signal of each channel after the filter process. The autocorrelation circuitry 134 performs autocorrelation calculation using reflected wave data generated by the generation circuitry 133, and the calculation circuitry 135 estimates a speed (V), power (P), and a variance (T) of the blood flow signal.

In addition, after a process of applying an MTI filter before the beam forming described above, like a conventional case, a process of further applying an MTI filter may be performed after the beam forming. Particularly, by applying an MTI filter according to the principal component analysis described above after beam forming brings further improvement of the elimination of clutters. While an MTI filter using the principal component analysis before beam forming has an effect of decreasing clutters according to a side lobe from a strong reflector, the influence of the main lobe becomes strong after the beam forming, and accordingly, the MTI filter using the principal component analysis has an effect of decreasing clutters according to the main lobe.

### Sixth Embodiment

While the first embodiment to the fifth embodiment described above have been described as methods for solving a saturation problem occurring at the time of imaging the blood flow information, the embodiment is not limited thereto. The first embodiment to the fourth embodiment among the embodiments described above, for example, may be also applied at the time of nonlinear imaging (THI) from a tissue or nonlinear imaging (CHI) from an imaging agent.

For example, in a case where a reflected wave signal is saturated in the reception circuitry, harmonics are generated. For this reason, in a filter method—harmonic imaging in which a reflected wave signal is acquired with a reception center frequency higher than a transmission center frequency, saturation is critical. In addition, in a pulse inversion method in which transmission is performed a plurality of times with the phase of the transmission pulse being changed, and signals are added, in a case where a reflected pulse signal is saturated, a signal from a strong reflector is lost to be darkened, and artifacts having a white circular arc shape as illustrated in FIG. 2 are generated in a side lobe area. In this way, at the time of performing the THI or the CHI, saturation of a reflected wave signal is a serious problem. For this reason, generally, the reception gain is set to be small so as not to saturate the reflected wave signal. However, by setting the reception gain to be small, the S/N ratio is degraded.

Based on such a situation, an ultrasonic diagnostic apparatus according a sixth embodiment decreases the influence of a saturated channel by applying one method according to one of the first embodiment to the fourth embodiment, and accordingly, a gain higher than that at the time of performing conventional THI/CHI can be set. The ultrasonic diagnostic apparatus according to the sixth embodiment transmits reflected wave data after beam forming to B-mode processing circuitry, and accordingly, a signal having an S/N ratio higher than that of the conventional case can be acquired while avoiding a problem occurring due to the saturation. The whole configuration of the ultrasonic diagnostic apparatus according to the sixth embodiment is the same as the whole configuration of the ultrasonic diagnostic apparatus 1 according to the first embodiment illustrated in FIG. 1 except for the application of the configuration of the reception circuitry according to the first embodiment or the second embodiment and the configuration of the reception circuitry and the beam former according to the third embodiment or the fourth embodiment.

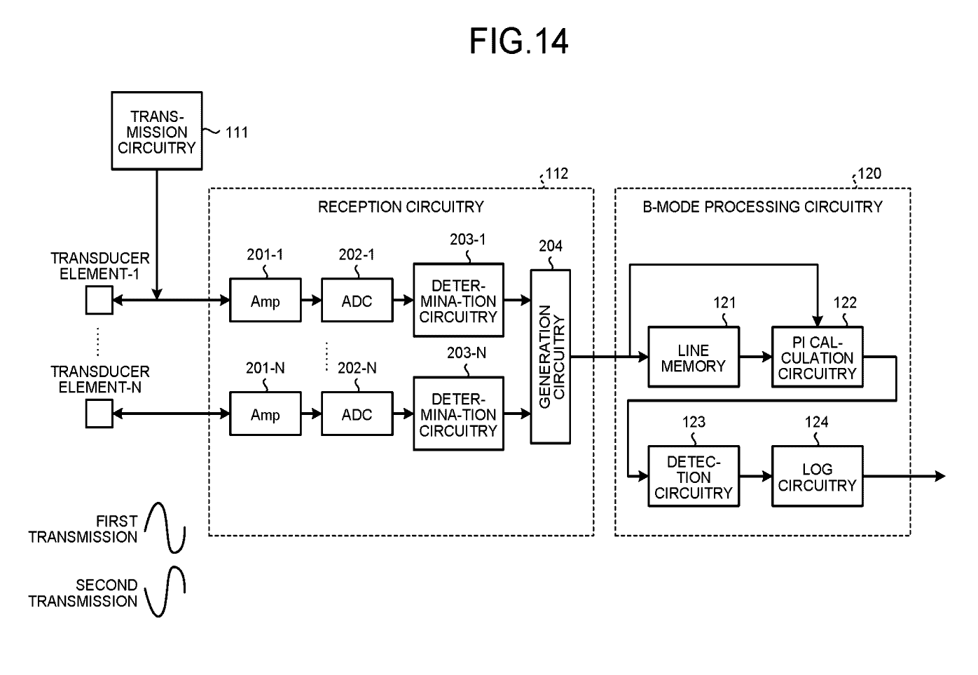

In the sixth embodiment, a case will be described in which harmonic imaging is performed using the pulse inversion (PI) method. FIG. 14 is a block diagram that illustrates an example of the configuration of reception circuitry and B-mode processing circuitry according to the sixth embodiment. FIG. 14 illustrates a case where reflected wave data of a harmonic component is extracted by the B-mode processing circuitry 120 by using reflected wave data generated by the reception circuitry 112. In such a case, for example, processing circuitry 170 causes an ultrasonic probe 11 to perform ultrasonic wave scanning having a first transmission ultrasonic wave and a second transmission ultrasonic wave acquired by inverting the phase of the first transmission ultrasonic wave as one set.

In the reception circuitry 112 according to the sixth embodiment, amplification circuitry 201 performs gain correction processing by amplifying a reflected wave signal (first reflected wave signal) of the first transmission ultrasonic wave and a reflected wave signal (second reflected wave signal) of the second transmission ultrasonic wave. An A/D converter 202 performs an A/D conversion of the gain-corrected first reflected wave signal and the gain-corrected second reflected wave signal. Then, the A/D converter 202 transmits the first reflected wave signal for which the A/D conversion has been performed and the second reflected wave signal for which the A/D conversion has been performed to determination circuitry 203.

In addition, in the reception circuitry 112, the determination circuitry 203 determines whether or not at least one of a reflected wave signal of the first transmission ultrasonic wave and a reflected wave signal of the second transmission ultrasonic wave, which have been received by each channel of the ultrasonic probe 11, is saturated. Then, the generation circuitry 204, by using an output signal for which a predetermined process corresponding to a result of the determination acquired by the determination circuitry 203 is performed, generates first reflected wave data for the reflected wave signal of the first transmission ultrasonic wave and second reflected wave data for the reflected wave signal of the second transmission ultrasonic wave. The generation circuitry 204 outputs the first reflected wave data and the second reflected wave data that have been generated to the B-mode processing circuitry 120.

The B-mode processing circuitry 120 includes: a line memory 121; PI calculation circuitry 122; detection circuitry 123; and log circuitry 124. The line memory 121 stores the first reflected wave data and the second reflected wave data generated by the generation circuitry 204. Then, the PI calculation circuitry 122 adds the reflected wave data of the first transmission ultrasonic wave and the reflected wave data of the second transmission ultrasonic wave generated by the generation circuitry 204 as a result of the ultrasonic wave scanning and extracts reflected wave data of a harmonic component. The detection circuitry 123 performs an envelope detection process for the reflected wave data. The log circuitry 124 performs logarithmic amplification for the reflected wave data, thereby generating B mode data. The B-mode processing circuitry 120 outputs the generated B mode data to the image generating circuitry 140. In this way, the image generating circuitry 140 generates an ultrasonic wave image by using signals acquired by extracting the harmonic component from the first reflected wave data and the second reflected wave data generated by the generation circuitry 204 as a result of the ultrasonic wave scanning.

In this way, the ultrasonic diagnostic apparatus according to the sixth embodiment can raise the gain to be higher than that of a conventional case in a case where a nonlinear signal is acquired from a tissue or an imaging agent without using the blood flow imaging method. For this reason, the S/N ratio is improved, and the sensitivity and the penetration can be improved.

Modified Example of Sixth Embodiment

In the sixth embodiment described above, while the case has been described in which reflected wave signals of a harmonic component are extracted by the B-mode processing circuitry at the time of performing harmonic imaging using the pulse inversion method, the embodiment is not limited thereto. For example, it may be configured such that PI calculation circuitry is arranged inside the reception circuitry, and reflected wave signals of a harmonic component are extracted by the reception circuitry. Thus, in a modified example of the sixth embodiment, a case will be described in which the reception circuitry includes the PI calculation circuitry.

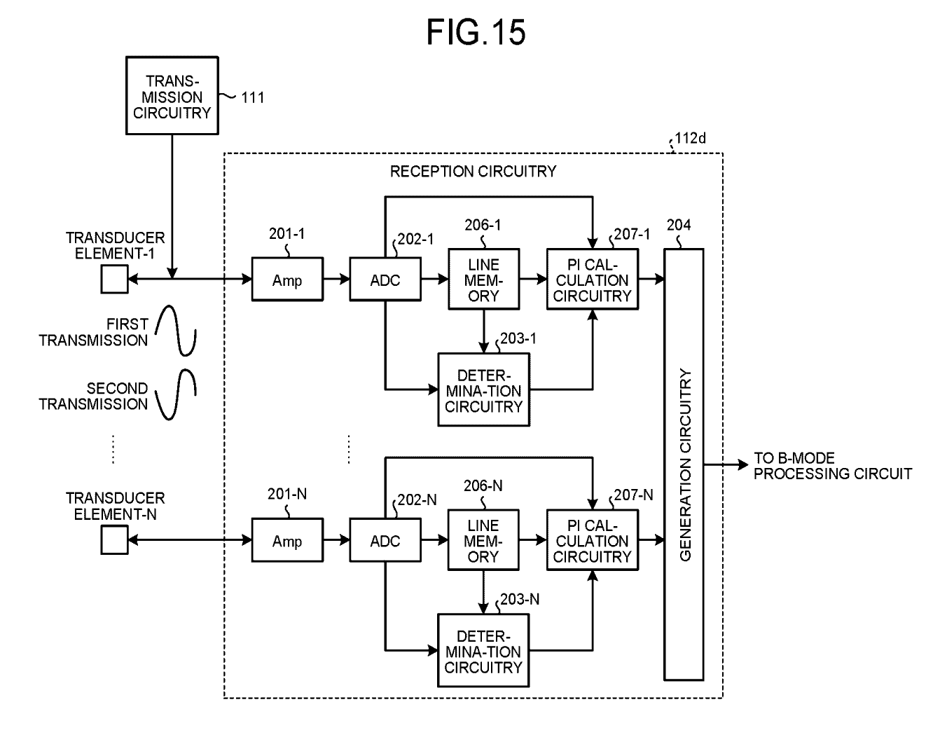

FIG. 15 is a block diagram that illustrates an example of the configuration of reception circuitry according to a modified example of the sixth embodiment. FIG. 15 illustrates a case where reflected wave signals of a harmonic component are extracted by reception circuitry 112d. In addition, in such a case, for example, the processing circuitry 170 causes the ultrasonic probe 11 to perform ultrasonic wave scanning having a first transmission ultrasonic wave and a second transmission ultrasonic wave acquired by inverting the phase of the first transmission ultrasonic wave as one set.

The reception circuitry 112d includes: amplification circuitry 201-1; an A/D converter 202-1; determination circuitry 203-1; a line memory 206-1; and PI calculation circuitry 207-1 as sub circuits used for processing a reflected wave signal received by Transducer element-1.

Similarly, the reception circuitry 112 includes: amplification circuitry 201-N; an A/D converter 202-N; determination circuitry 203-N; a line memory 206-N; and PI calculation circuitry 207-N as sub circuits used for processing a reflected wave signal received by Transducer element-N. Here, in a case where the amplification circuitry 201-1 and the amplification circuitry 201-N do not need to be discriminated from each other, the amplification circuitry will be described as amplification circuitry 201. In addition, in a case where the A/D converter 202-1 and the A/D converter 202-N do not need to be discriminated from each other, the A/D converter will be described as an A/D converter 202, and, in a case where the determination circuitry 203-1 and the determination circuitry 203-N do not need to be discriminated from each other, the determination circuitry will be described as determination circuitry 203. Furthermore, in a case where the line memory 206-1 and the line memory 206-N do not need to be discriminated from each other, the line memory will be described as a line memory 206, and, in a case where the PI calculation circuitry 207-1 and the PI calculation circuitry 207-N do not need to be discriminated from each other, the PI calculation circuitry will be described as PI calculation circuitry 207. In other words, in the reception circuitry 112, the amplification circuitry 201, the A/D converter 202, the determination circuitry 203, the line memory 206, and the PI calculation circuitry 207 are disposed for each transducer element (channel).

The amplification circuitry 201 performs a gain correction process by amplifying a reflected wave signal for each channel. For example, the amplification circuitry 201 performs the gain correction process by amplifying a reflected wave signal (first reflected wave signal) of the first transmission ultrasonic wave and a reflected wave signal (second reflected wave signal) of the second transmission ultrasonic wave. The A/D converter 202 performs an A/D conversion of the gain-corrected reflected wave signals. For example, the A/D converter 202 performs an A/D conversion of the gain-corrected first reflected wave signal and the gain-corrected second reflected wave signal. Then, the A/D converter 202 transmits the A/D-converted first reflected wave signal and the A/D-converted second reflected wave signal to the line memory 206. The line memory 206 stores the A/D-converted first reflected wave signal and the A/D-converted second reflected wave signal.

In the reception circuitry 112d, the determination circuitry 203 determines whether or not at least one of a reflected wave signal of the first transmission ultrasonic wave and a reflected wave signal of the second transmission ultrasonic wave, which have been received by each channel of the ultrasonic probe 11, is saturated. Then, the PI calculation circuitry 207 adds the reflected wave signal of the first transmission ultrasonic wave and the reflected wave signal of the second transmission ultrasonic wave and extracts reflected wave signals of a harmonic component. Here, the PI calculation circuitry 207 outputs an output signal in which the value of the reflected wave signal of the harmonic component is “0” in a case where saturation is determined by the determination circuitry 203 and outputs a reflected wave signal of a harmonic component that is extracted after the reflected wave signal of the first transmission ultrasonic wave and the reflected wave signal of the second transmission ultrasonic wave are added as an output signal in a case where no saturation is determined by the determination circuitry 203. Then, the generation circuitry 204 generates reflected wave data by using the output signal output by the PI calculation circuitry 207.

The B-mode processing circuitry 120 generates B mode data based on the reflected wave data generated by the generation circuitry 204. Then, the B-mode processing circuitry 120 outputs the generated B mode data to the image generating circuitry 140. In this way, the image generating circuitry 140 generates an ultrasonic wave image by using the signal acquired by extracting the harmonic component from the reflected wave data generated by the generation circuitry 204 as a result of the ultrasonic wave scanning.

Filter Method—Harmonic Imaging

In the sixth embodiment described above, while the case has been described in which harmonic imaging is performed using the pulse inversion method, the embodiment is not limited thereto. For example, in the ultrasonic diagnostic apparatus according to the sixth embodiment, the filter method—harmonic imaging may be performed. Thus, a case will be described in which the ultrasonic diagnostic apparatus according to the sixth embodiment performs the filter method—harmonic imaging. Here, a case will be described in which the reception circuitry 112 according to the first embodiment is applied. For example, the processing circuitry 170 causes the ultrasonic probe 11 to perform ultrasonic wave scanning acquiring the information of a tissue shape. In the reception circuitry 112, the determination circuitry 203 determines whether or not a reflected wave signal received by each channel of the ultrasonic probe 11 is saturated. The generation circuitry 204 generates reflected wave data by performing a phasing addition process using an output signal for which a predetermined process corresponding to a result of the determination acquired by the determination circuitry 203 has been performed. Subsequently, the B-mode processing circuitry 120 extracts a harmonic component from the reflected wave data by performing a filter process. Then, the image generating circuitry 140 generates an ultrasonic wave image by using the harmonic component extracted by the B-mode processing circuitry 120. In other words, the image generating circuitry 140, as a result of ultrasonic wave scanning, generates an ultrasonic wave image by using a signal acquired by extracting a harmonic component from the reflected wave data generated by the generation circuitry 204.

### Other Embodiment

The embodiment is not limited to the embodiments described above.

The first embodiment to the sixth embodiment described above may be combined and used.

In the first embodiment to the sixth embodiment described above, the process performed by the ultrasonic diagnostic apparatus may be performed by an apparatus other than the ultrasonic diagnostic apparatus. For example, a signal of each channel CH before beam forming is stored in the storage circuitry 160 from the reception circuitry 112 through a bus. Then, the apparatus other than the ultrasonic diagnostic apparatus, for example, may display an image by reading a signal of each channel CH before beam forming after the stop of ultrasonic wave scanning, outputting data using the method described in the first embodiment to the sixth embodiment described above, and performing B-mode processing and color Doppler processing. For example, the signal processing unit includes determination circuitry and a generation circuitry. The determination circuitry determines whether or not a reflected wave signal received by each channel of the ultrasonic probe 11 is saturated. The generation circuitry generates reflected wave data by performing a phasing addition process using an output signal for which a predetermined process corresponding to a result of the determination acquired by the determination circuitry has been performed.

In the description of the embodiments described above, each constituent element of each apparatus illustrated in the drawing is functional and conceptual, and it is not necessary to physically configure each apparatus as illustrated in the drawing. In other words, a specific form of separation/integration of each apparatus is not limited to that illustrated in the drawing, and the whole or a part of each apparatus may be functionally or physically distributed/integrated in an arbitrary unit in accordance with various loads, the use status, and the like. In addition, the entirety or an arbitrary part of each processing function performed in each apparatus may be realized by a CPU and a program that is interpreted and executed by the CPU or may be realized by hardware using a wired logic.

In addition, the control method described in the embodiments described above may be realized by executing a control program prepared in advance using a computer such as a personal computer or a workstation. This control program may be distributed through a network such as the Internet. In addition, this control program may be recorded on a computer-readable recording medium such as a hard disk, a flexible disk (FD), a CD-ROM, an MO, or a DVD and can be executed by being read by a computer from the recording medium.

According to at least one of the embodiments described above, an artifact according to a strong reflector can be decreased.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
determination circuitry configured to determine whether or not a reflected wave signal received by each channel of an ultrasonic probe is saturated; and
generation circuitry configured to generate reflected wave data through a phasing addition process using an output signal for which a predetermined process corresponding to a result of the determination acquired by the determination circuitry is performed.

2. The ultrasonic diagnostic apparatus according to claim 1,
wherein the determination circuitry, in a case where a value of a reflected wave signal is predetermined threshold or more, determines that the reflected wave signal is saturated, and
the generation circuitry generates the reflected wave data by using an output value acquired by multiplying a reflected wave signal of a saturated channel by a predetermined coefficient having a value of one or less as the output signal.

3. The ultrasonic diagnostic apparatus according to claim 1, further comprising:
first correction circuitry configured to acquire a first corrected signal from a reflected wave signal by correcting the reflected wave signal of each channel using a first gain value; and
second correction circuitry configured to acquire a second corrected signal from a reflected wave signal by correcting the reflected wave signal of each channel using a second gain value less than the first gain value,
wherein the determination circuitry determines whether or not the reflected wave signal of each channel is saturated by comparing the first corrected signal with the second corrected signal.

4. The ultrasonic diagnostic apparatus according to claim 3, further comprising switching control circuitry configured to perform switching between a first state in which the ultrasonic probe is connected to the first correction circuitry and the second correction circuitry and a second state in which the ultrasonic probe is connected to one of the first correction circuitry and the second correction circuitry in correspondence with the number of channels used for ultrasonic wave scanning.

5. The ultrasonic diagnostic apparatus according to claim 1, further comprising correction circuitry configured to, when a gain value of a received reflected wave signal is corrected in units of frames, alternately correct a gain value for each adjacent frame using a first gain value and a second gain value less than the first gain value and acquire a first corrected signal and a second corrected signal from the reflected wave signal in units of frames,
wherein the determination circuitry determines whether or not the reflected wave signal in units of frames is saturated by comparing the first corrected signal with the second corrected signal.

6. The ultrasonic diagnostic apparatus according to claim 3,
wherein the determination circuitry determines that a reflected wave signal is not saturated in a case where an absolute value of a difference between the first corrected signal and the second corrected signal is a predetermined threshold or less, and
the generation circuitry generates the reflected wave data by using the first corrected signal as the output signal in a case where the reflected wave signal is determined not to be saturated or by using a signal acquired by mixing the first corrected signal and the second corrected signal at a predetermined ratio as the output signal in a case where the reflected wave signal is determined to be saturated.

7. The ultrasonic diagnostic apparatus according to claim 4,
wherein the determination circuitry determines that a reflected wave signal is not saturated in a case where an absolute value of a difference between the first corrected signal and the second corrected signal is a predetermined threshold or less, and
the generation circuitry generates the reflected wave data by using the first corrected signal as the output signal in a case where the reflected wave signal is determined not to be saturated or by using a signal acquired by mixing the first corrected signal and the second corrected signal at a predetermined ratio as the output signal in a case where the reflected wave signal is determined to be saturated.

8. The ultrasonic diagnostic apparatus according to claim 5,
wherein the determination circuitry determines that a reflected wave signal is not saturated in a case where an absolute value of a difference between the first corrected signal and the second corrected signal is a predetermined threshold or less, and
the generation circuitry generates the reflected wave data by using the first corrected signal as the output signal in a case where the reflected wave signal is determined not to be saturated or by using a signal acquired by mixing the first corrected signal and the second corrected signal at a predetermined ratio as the output signal in a case where the reflected wave signal is determined to be saturated.

9. The ultrasonic diagnostic apparatus according to claim 1, wherein the generation circuitry, as a result of ultrasonic wave scanning, in a case where reflected wave signals of a plurality of adjacent channels corresponding to a predetermined number or more from among the reflected wave signals from the channels are saturated, generates the reflected wave data by using output values acquired by multiplying the reflected wave signals of the plurality of channels by a predetermined coefficient having a value of one or less as the output signal.

10. The ultrasonic diagnostic apparatus according to claim 1, further comprising control circuitry configured to cause the ultrasonic probe to perform first ultrasonic wave scanning acquiring information relating to a motion of a moving body within a first scanning range and cause the ultrasonic probe to perform ultrasonic wave scanning of each of a plurality of divided ranges acquired by dividing a second scanning range as second ultrasonic wave scanning acquiring information of a tissue shape within the second scanning range in a time divisional manner during the first ultrasonic wave scanning,
wherein the determination circuitry determines whether or not a reflected wave signal received by each channel of the ultrasonic probe is saturated in accordance with at least the first ultrasonic wave scanning.

11. The ultrasonic diagnostic apparatus according to claim 3, further comprising:
control circuitry configured to cause the ultrasonic probe to perform ultrasonic wave scanning acquiring information relating to a motion of a moving body, and
image generating circuitry configured to generate an image including information of a tissue shape by using the second gain value.

12. The ultrasonic diagnostic apparatus according to claim 4, further comprising:
control circuitry configured to cause the ultrasonic probe to perform ultrasonic wave scanning acquiring information relating to a motion of a moving body, and
image generating circuitry configured to generate an image including information of a tissue shape by using the second gain value.

13. The ultrasonic diagnostic apparatus according to claim 5, further comprising:
control circuitry configured to cause the ultrasonic probe to perform ultrasonic wave scanning acquiring information relating to a motion of a moving body, and
image generating circuitry configured to generate an image including information of a tissue shape by using the second gain value.

14. The ultrasonic diagnostic apparatus according to claim 1, further comprising:
control circuitry configured to causes the ultrasonic probe to perform ultrasonic wave scanning acquiring information relating to a tissue shape; and
image generating circuitry configured to generate an image by extracting a harmonic component from the reflected wave data generated by the generation circuitry as a result of the ultrasonic wave scanning.

15. The ultrasonic diagnostic apparatus according to claim 1, further comprising:
control circuitry configured to cause the ultrasonic probe to perform ultrasonic wave scanning having a first transmission ultrasonic wave and a second transmission ultrasonic wave acquired by inverting a phase of the first transmission ultrasonic wave as one set; and
calculation circuitry configured to add the reflected wave data of the first transmission ultrasonic wave and the reflected wave data of the second transmission ultrasonic wave generated by the generation circuitry as a result of the ultrasonic wave scanning and extract reflected wave data of a harmonic component.

16. The ultrasonic diagnostic apparatus according to claim 1, further comprising:
control circuitry configured to cause the ultrasonic probe to perform ultrasonic wave scanning having a first transmission ultrasonic wave and a second transmission ultrasonic wave acquired by inverting a phase of the first transmission ultrasonic wave as one set; and
calculation circuitry configured to add a reflected wave signal of the first transmission ultrasonic wave and a reflected wave signal of the second transmission ultrasonic wave and extract a reflected wave signal of a harmonic component,
wherein the determination circuitry determines whether or not at least one of the reflected wave signal of the first transmission ultrasonic wave and the reflected wave signal of the second transmission ultrasonic wave, which are received by each channel of the ultrasonic probe, is saturated,
the calculation circuitry outputs an output signal of which a value of the reflected wave signal of the harmonic component is zero in a case where saturation is determined by the determination circuitry and outputs a reflected wave signal of the harmonic component extracted from a signal acquired by adding the reflected wave signal of the first transmission ultrasonic wave and the reflected wave signal of the second transmission ultrasonic wave as the output signal in a case where no saturation is determined by the determination circuitry, and
the generation circuitry generates the reflected wave data by using the output signal output by the calculation circuitry.

17. The ultrasonic diagnostic apparatus according to claim 1, further comprising control circuitry configured to cause the ultrasonic probe to perform ultrasonic wave scanning transmitting a plane wave.

18. The ultrasonic diagnostic apparatus according to claim 17, wherein the control circuitry causes the ultrasonic probe to perform ultrasonic wave scanning receiving reflected wave signals in a plurality of scanning lines.

19. A signal processing apparatus comprising:
determination circuitry configured to determine whether or not a reflected wave signal received by each channel of an ultrasonic probe is saturated; and
generation circuitry configured to generate reflected wave data through a phasing addition process using an output signal for which a predetermined process corresponding to a result of the determination acquired by the determination circuitry is performed.
